(19) United States
(12) Patent Application Publication
(10) Pub. No.: US 2021/0230131 A1
(43) Pub. Date: Jul. 29, 2021

(54) POLYAMINO BIARYL COMPOUNDS AND THEIR USE

(71) Applicants: INSERM (INSTITUT NATIONAL DE LA SANTÉ ET DE LA RECHERCHE MÉDICALE), Paris (FR); UNIVERSITE DE LILLE, Lille (FR); CENTRE HOSPITALIER REGIONSL ET UNIVERSITAIRE DE LILLE (CHRU), Lille (FR)

(72) Inventors: Patricia MELNYK, Lille (FR); Marie TAUTOU, Lille (FR); Caroline EVRARD, Lille (FR); Florian DESCAMPS, Lille (FR); Jamal EL BALKALI, Lille (FR); Marion GAY, Neuville-Sur-Oise (FR); Nicolas RENAULT, Lille (FR); Nicolas SERGEANT, Lille (FR); Pascal CARATO, Poitiers (FR); Valérie VINGTDEUX, Lille (FR)

(21) Appl. No.: 17/257,121

(22) PCT Filed: Jul. 10, 2019
(86) PCT No.: PCT/EP2019/068520

§ 371 Date: Dec. 30, 2020

(30) Foreign Application Priority Data
Jul. 11, 2018 (EP) 18305932.8

(51) Int. Cl. C07D 295/13; A61P 25/28; C07D 207/333; C07D 295/088

(52) U.S. Cl. CPC C07D 295/13; C07D 295/088; C07D 207/333; A61P 25/28

(57) ABSTRACT

The present invention is directed to novel compounds of Formula I and pharmaceutically acceptable salts or solvates thereof, and their use.

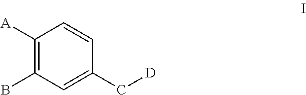

The present invention relates to novel polyamino biaryl compounds including their pharmaceutically acceptable salts and solvates which are inhibitors of Aβ peptides production and/or stabilizers of αCTFs and AICD expression and are useful as therapeutic compounds, particularly in the treatment and/or prevention of diseases involving formation of amyloid plaques and/or where a dysfunction of the APP metabolism occurs.

### BACKGROUND OF THE INVENTION

Alzheimer's disease (AD) is a progressive neurodegenerative disorder characterized by a slow decline of cognitive and behavioral abilities. It affects brain functions, including short-term memory loss, inability to reason, and the deterioration of language and the ability to care for oneself. An estimated 3% of people between the ages of 65 and 74 have Alzheimer's disease, rising to half those age 85 and over.

Two neuropathological lesions provide the definite diagnosis of AD: neurofibrillary tangles and amyloid deposits. Neurofibrillary tangles are characterized by intraneuronal accumulation and aggregation of abnormally modified isoforms of the microtubule-associated tau proteins. Parenchymal amyloid deposits are composed of amyloid-β (Aβ) peptides, which derive from its precursor, the so-called amyloid precursor protein (APP). Current treatments for AD remain symptomatic and provide only limited benefits to the patients. Therefore, alternative therapeutic options are urgently needed and over the past few years, much effort has been dedicated to the development of disease-modifying therapeutics. One such strategy consists of reducing the levels of toxic Aβ peptides by targeting APP processing and more specifically, secretases that cleave APP down to toxic Aβ peptides (mainly Aβ₁₋₄₂). Indeed, the metabolism of APP involves the action of several secretases and is sub-cellularly compartmentalized. APP is known to undergo proteolytic processing through two main pathways: the non-amyloidogenic pathway, initiated by α-secretase cleavage and the amyloidogenic pathway initiated by β-secretase cleavage. Both secretase-mediated steps release soluble ectodomains of APP (sAPPα and sAPPβ) and the membrane-bound carboxyl-terminal fragments (APP-CTFs), known as αCTF and βCTF. The latter are further cleaved by γ-secretase to give peptide p3 and Aβ peptide, respectively, along with the APP intracellular domain (AICD).

Shifting of APP processing towards the non-amyloidogenic pathway can be achieved either by blocking the amyloidogenic pathway or by promoting the non-amyloidogenic pathway. Because amyloidogenic processing is initiated by the cleavage of APP by β-secretase (β-site amyloid-precursor-protein-cleaving enzyme 1, BACE-1), this protease has been suggested as an attractive target to lower Aβ levels and the resulting amyloid plaques. Recently, a mutation in APP (APP-A673T) that protects against the development of AD and age-related cognitive decline has been identified, providing further evidence that reducing β-secretase cleavage of APP may protect against AD (Jonsson, T. et al., Nature 2012, 488, 96; Maloney, J. A. et al., J. Biol. Chem. 2014, 289, 30990-31000). Most efforts regarding β-secretase inhibitors have been focused on active site binders, and although β-secretase inhibitor drug development has proven challenging, several inhibitors are currently in clinical development. Given that β-secretase has multiple substrates involved in important cellular and tissue functions, its inhibition may produce mechanism-based side effects. As an alternative, a recent study showed that the anticancer drug Gleevec lowers Aβ levels through indirect inhibition of β-secretase processing of APP (Ben Halima, S. et al., Cell Rep. 2016, 14 (9), 2127-2141). Another recent study taking advantage of the compartmentalization of APP processing, showed that β-secretase inhibitors can be designed to specifically inhibit APP processing in the endosomal compartment with no effect on non-amyloid substrates (Netzer, W. J. et al., Proc. Natl. Acad. Sci. 2017, 114 (6), 1389-1394). Indeed, the endosomal/lysosomal compartments are known to be of importance for APP metabolism, APP degradation as well as for the production of Aβ peptides (Ghavami, S. et al., Prog. Neurobiol. 2014, 112, 24-49; Correia, S. C. et al., DNA Cell Biol. 2015, 34, 261-273; Peric, A. et al., Acta Neuropathol. 2015, 129, 363-381). In this context, the lysosomotropic agent, chloroquine (CQ) has been reported to inhibit Aβ peptide production.

WO 2006/051489 discloses the use of 1,4-bis(3-aminoalkyl)piperazine derivatives for the treatment of neurodegenerative diseases, wherein said derivatives may be used to rectify the metabolism of the amyloid protein precursor (APP).

WO 2011/073322 discloses the use of 7-chloro-quinolin-4-amine compounds for the prevention or treatment of diseases involving formation of amyloid plaques and/or where a dysfunction of the APP metabolism occurs.

However, there is still a need for new compounds having the ability to inhibit Aβ peptides secretion and to stabilize αCTFs and AICD expression and that are of therapeutic value for the treatment and/or prevention of diseases involving formation of amyloid plaques and/or where a dysfunction of the APP metabolism occurs.

### SUMMARY OF THE INVENTION

The inventors have now succeeded in developing novel compounds based on a biaryl scaffold bearing amino side chains. These compounds have the advantage of inhibiting the production of Aβ peptides and/or stabilizing αCTFs and AICD expression with a higher efficacy than chloroquine.

The invention therefore relates to compounds of general Formula I, their pharmaceutically acceptable salts and solvates as well as methods of use of such compounds or compositions comprising such compounds as inhibitors of production of Aβ peptides and/or stabilizers of αCTFs and AICD expression.

In a general aspect, the invention provides compounds of general Formula I:

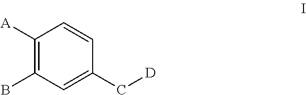

and pharmaceutically acceptable salts and solvates thereof,

wherein

A is H or a group of formula

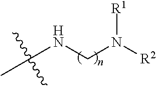

wherein

R¹ and R² are independently selected from C₁-C₆-alkyl and C₁-C₆-haloalkyl, or

R¹ and R² form together with the nitrogen atom they are attached to a 6- or 7-membered heterocyclyl group, which optionally contains one or more other heteroatoms, and wherein the resulting heterocyclic moiety is optionally substituted by one or more substituents independently selected from C₁-C₄-alkyl, halogen and C₁-C₄-haloalkyl;

n is an integer from 1 to 6;

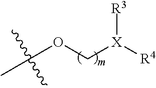

B is H or a group of formula wherein

X is N or CH;

R³ and R⁴ are independently selected from C1-C6-alkyl and C1-C6-haloalkyl, or

R³ and R⁴ form together with X a 6-membered cycloalkyl group or a 6- or 7-membered heterocyclyl group, which optionally contains one or more other heteroatoms, and wherein the resulting cyclic or heterocyclic moiety is optionally substituted by one or more substituents independently selected from C1-C4-alkyl, halogen, C1-C4-haloalkyl;

m is an integer from 1 to 6;

C is a 5- or 6-membered aryl or heteroaryl group;

D is H or a group of formula

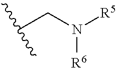

wherein

R⁵ and R⁶ are independently selected from C1-C6-alkyl and C1-C6-haloalkyl, or

R⁵ and R⁶ form together with the nitrogen atom they are attached to a 6- or 7-membered heterocyclyl group, which optionally contains one or more other heteroatom, and wherein the resulting heterocyclic moiety is optionally substituted by one or more substituents independently selected from C1-C4-alkyl, halogen, C1-C4-haloalkyl; and

D is located at any free position of group C;

with the proviso that at least two groups amongst groups A, B and D are not H.

In another aspect, the present invention provides a pharmaceutical composition comprising at least one compound according to the invention or a pharmaceutically acceptable salt or solvate thereof and at least one pharmaceutically acceptable carrier, diluent, excipient and/or adjuvant.

The invention also relates to the use of the above compounds or their pharmaceutically acceptable salts and solvates thereof as inhibitors of Aβ peptides production and/or stabilizers of αCTFs and AICD expression.

The invention further provides the use of a compound according to the invention or a pharmaceutically acceptable salt or solvate thereof as a medicament. Preferably, the medicament is used for the treatment and/or prevention of diseases involving formation of amyloid plaques and/or where a dysfunction of the APP metabolism occurs.

### DETAILED DESCRIPTION OF THE INVENTION

As detailed above, the invention relates to compounds of Formula I, as well as their pharmaceutically acceptable salts and solvates.

Preferred compounds of Formula I and pharmaceutically acceptable salts and solvates thereof are those wherein one or more of A and B, C and D are defined as follows:

A is H or a group of formula

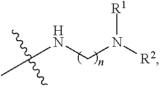

preferably A a group of formula

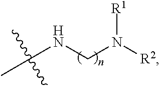

wherein R¹ and R² are independently selected from C1-C6-alkyl and C1-C6-haloakyl, preferably R¹ and R² are independently selected from C1-C4-alkyl and C1-C4-haloakyl, more preferably R¹ and R² are independently selected from C1-C2-alkyl, even more preferably R¹ and R² are both methyl, or

R¹ and R² form together with the nitrogen atom they are attached to a 6- or 7-membered heterocyclyl group, preferably a 6-membered heterocyclyl group, which optionally contains one or more other heteroatoms, and wherein the resulting heterocyclic moiety is optionally substituted by one or more substituents independently selected from C1-C4-alkyl, halogen and C1-C4-haloalkyl; preferably R¹ and R² form together with the nitrogen atom they are attached to a non-aromatic 6-membered heterocyclyl group, which optionally contains another heteroatom selected from oxygen and nitrogen, and wherein the resulting heterocyclic moiety is optionally substituted by one or more substituents independently selected from C1-C4-alkyl, preferably C1-C2-alkyl; more preferably R¹ and R² form together with the nitrogen atom they are attached to a non-aromatic 6-membered heterocyclyl group selected from piperidinyl, morpholinyl and N-methylpiperazinyl; still more preferably, R¹ and R² form together with the nitrogen atom they are attached to a piperidinyl group;

n is an integer from 1 to 6, preferably from 2 to 4, more preferably n is 2 or 3, even more preferably n is 3;

B is H or a group of formula

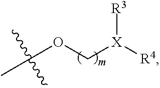

preferably B is a group of formula

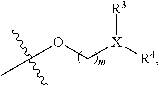

wherein

X is N or CH, preferably X is N;

R³ and R⁴ are independently selected from C1-C6-alkyl, preferably C1-C4-alkyl, more preferably C1-C2-alkyl, even more preferably R³ and R⁴ are both methyl, or

R³ and R⁴ form together with X a 6-membered cycloalkyl group or a 6- or 7-membered heterocyclyl group, preferably a 6-membered heterocyclyl group, which optionally contains one or more other heteroatom, and wherein the resulting cyclic or heterocyclic moiety is optionally substituted by one or more substituents independently selected from C1-C4-alkyl, halogen and C1-C4-haloalkyl; preferably R³ and R⁴ form together with X a non-aromatic 6-membered cycloalkyl group or a 6-membered heterocyclyl group, which optionally contains another heteroatom selected from oxygen and nitrogen, and wherein the resulting cyclic or heterocyclic moiety is optionally substituted by one or more substituents independently selected from C1-C4-alkyl, preferably C1-C2-alkyl; more preferably R³ and R⁴ form together with X a non-aromatic 6-membered cycloalkyl or heterocyclyl group selected from piperidinyl, morpholinyl, N-methylpiperazinyl and cyclohexyl; still more preferably, R³ and R⁴ form together with X a cyclohexyl group or a piperidinyl group;

m is an integer from 1 to 6, preferably from 2 to 4, more preferably n is 2 or 3, even more preferably n is 2;

C is a 5- or 6-membered aryl or heteroaryl group, preferably C is a 5- or 6-membered aryl or heteroaryl group containing a nitrogen atom, more preferably C is selected from phenyl, pyridyl, pyrazinyl, pyrimidinyl, pyridazinyl, 1,2,3-triazinyl, 1,2,4-triazinyl, 1,3,5-triazinyl, pyrrolyl, imidazolyl, pyrazolyl, oxazolyl, isoxazolyl and thiazolyl, still more preferably, C is a 6-membered aryl group or a 5-membered heteroaryl group containing a nitrogen atom, even more preferably, C is phenyl or pyrrolyl;

D is H or a group of formula

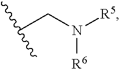

preferably D is a group of formula

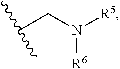

wherein

R⁵ and R⁶ are independently selected from C1-C6-alkyl, preferably C1-C4-alkyl, more preferably C1-C2-alkyl, even more preferably R³ and R⁴ are both methyl, or R⁵ and R⁶ form together with the nitrogen atom they are attached to a 6- or 7-membered heterocyclyl group, preferably a 6-membered heterocyclyl group, which optionally contains one or more other heteroatom, and wherein the resulting heterocyclic moiety is optionally substituted by one or more substituents independently selected from C1-C4-alkyl, halogen and C1-C4-haloalkyl; preferably, R⁵ and R⁶ form together with the nitrogen atom they are attached to a non-aromatic 6- or 7-membered heterocyclyl group, preferably a 6-membered heterocyclyl group, which optionally contains another heteroatom selected from oxygen and nitrogen, and wherein the resulting heterocyclic moiety is optionally substituted by one or more substituents independently selected from C1-C4-alkyl, preferably C1-C2-alkyl; more preferably, R⁵ and R⁶ form together with the nitrogen atom they are attached to a non-aromatic 6- or 7-membered heterocyclyl group selected from piperidinyl, morpholinyl, N-methylpiperazinyl, and homopiperazinyl; still more preferably, R⁵ and R⁶ form together with the nitrogen atom they are attached to a non-aromatic 6-membered heterocyclyl group selected from piperidinyl, morpholinyl and N-methylpiperazinyl; and

D is located at any free position of group C;

with the proviso that at least two groups amongst groups A, B and D are not H.

In one embodiment, C is a 6-membered aryl group, preferably phenyl, and D is a group of formula

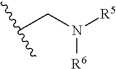

located at any free position of group C.

In one embodiment, A is H and C is a 6-membered aryl group, preferably phenyl, and D is a group of formula

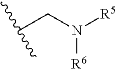

located at any free position of group C, preferably at position 2 of group C.

In one embodiment, B is H and C is a 6-membered aryl group, preferably phenyl, and D is a group of formula

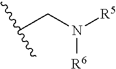

located at any free position of group C, preferably at position 2 of group C.

In one embodiment, C is a 5-membered heteroaryl group, preferably a 5-membered heteroaryl group containing a nitrogen atom, more preferably a pyrrolyl group, even more preferably a pyrrolyl group linked to the central phenyl group through the nitrogen atom, and D is a group of formula

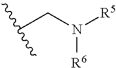

located at any free position of group C, preferably at position 3 of group C.

In fact, and without wanting to be tied to any theory whatsoever, the inventors think that the ability to both inhibit the secretion of Aβ peptides and/or to promote AICD and αCTFs stability of the compounds according to the invention is obtained thanks to the presence of two or three tertiary amines.

In one embodiment, preferred compounds of Formula I are those of Formula II

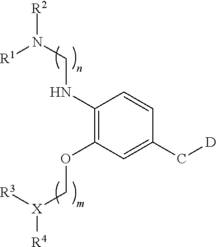

and pharmaceutically acceptable salts and solvates thereof,

wherein

X, R¹, R², R³, R⁴, n, m, C and D are as defined above with respect to Formula I and any of its embodiments;

with the proviso that D is not H when X is CH.

In one embodiment, preferred compounds of Formula II are those of Formula IIa:

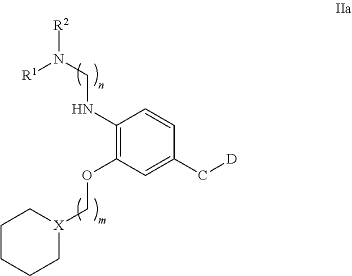

and pharmaceutically acceptable salts and solvates thereof,

wherein

X, R¹, R², n, m, C and D are as defined above with respect to Formula I and any of its embodiments;

with the proviso that D is not H when X is CH.

In one embodiment, preferred compounds of Formula II are those of Formula IIb:

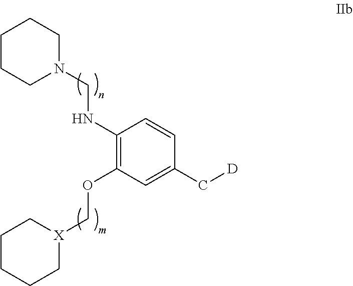

and pharmaceutically acceptable salts and solvates thereof,

wherein

X, n, m, C and D are as defined above with respect to Formula I and any of its embodiments;

with the proviso that D is not H when X is CH.

In one embodiment, preferred compounds of Formula I are those of Formula III:

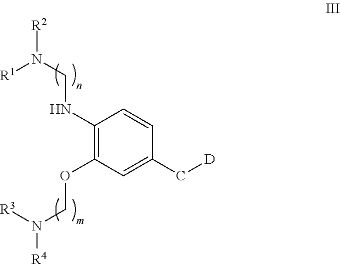

and pharmaceutically acceptable salts and solvates thereof,

wherein

R¹, R², R³, R⁴, n, m, C and D are as defined above with respect to Formula I and any of its embodiments;

In one embodiment, preferred compounds of Formula III are those of Formula IIIa:

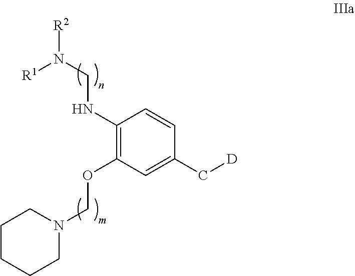

and pharmaceutically acceptable salts and solvates thereof,

wherein

R¹, R², n, m, C and D are as defined above with respect to Formula I and any of its embodiments.

In one embodiment, preferred compounds of Formula III are those of Formula IIIb:

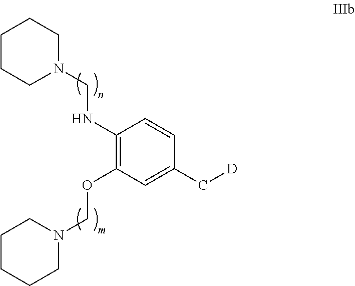

and pharmaceutically acceptable salts and solvates thereof,

wherein

n, m, C and D are as defined above with respect to Formula I and any of its embodiments.

In one embodiment, preferred compounds of Formula I are those of Formula IV:

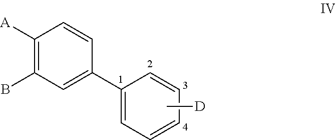

and pharmaceutically acceptable salts and solvates thereof,

wherein

A, B and D are as defined above with respect to Formula I and any of its embodiments.

In one embodiment, D is a group of formula

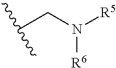

located at position 2 of the phenyl ring, wherein R⁵ and R⁶ are as defined above with respect to Formula I and any of its embodiments.

In one embodiment, D is a group of formula

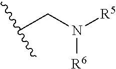

located at position 3 of the phenyl ring, wherein R⁵ and R⁶ are as defined above with respect to Formula I and any of its embodiments.

In one embodiment, D is a group of formula

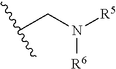

located at position 4 of the phenyl ring, wherein R⁵ and R⁶ are as defined above with respect to Formula I and any of its embodiments.

In one embodiment, A is H and D is a group of formula

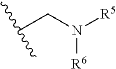

located at position 2 of the phenyl ring, wherein R⁵ and R⁶ are as defined above with respect to Formula I and any of its embodiments.

In one embodiment, B is H and D is a group of formula

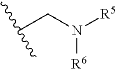

located at position 2 of the phenyl ring, wherein R⁵ and R⁶ are as defined above with respect to Formula I and any of its embodiments.

In one embodiment, preferred compounds of Formula I are those of Formula IVa:

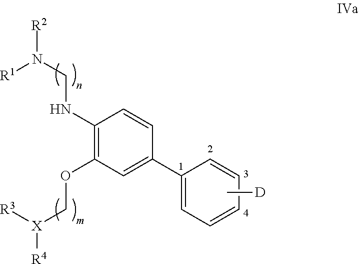

and pharmaceutically acceptable salts and solvates thereof,

wherein

X, R¹, R², R³, R⁴, n, m and D are as defined above with respect to Formula I and any of its embodiments.

In one embodiment, D is a group of formula

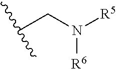

located at position 2 of the phenyl ring, wherein R⁵ and R⁶ are as defined above with respect to Formula I and any of its embodiments.

In one embodiment, D is a group of formula

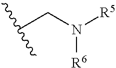

located at position 3 of the phenyl ring, wherein R⁵ and R⁶ are as defined above with respect to Formula I and any of its embodiments.

In one embodiment, D is a group of formula

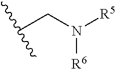

located at position 4 of the phenyl ring, wherein R⁵ and R⁶ are as defined above with respect to Formula I and any of its embodiments.

In one embodiment, preferred compounds of Formula IV are those of Formula IVb:

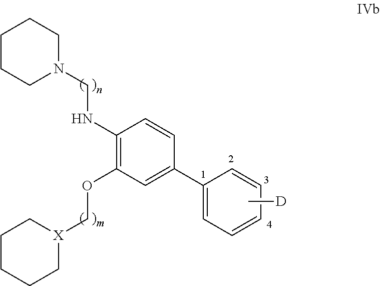

and pharmaceutically acceptable salts and solvates thereof,

wherein

X, n, m and D are as defined above with respect to Formula I and any of its embodiments.

In one embodiment, D is a group of formula

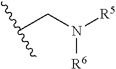

located at position 2 of the phenyl ring, wherein R⁵ and R⁶ are as defined above with respect to Formula I and any of its embodiments.

In one embodiment, D is a group of formula

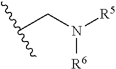

located at position 3 of the phenyl ring, wherein R⁵ and R⁶ are as defined above with respect to Formula I and any of its embodiments.

In one embodiment, D is a group of formula

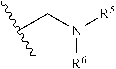

located at position 4 of the phenyl ring, wherein R⁵ and R⁶ are as defined above with respect to Formula I and any of its embodiments.

In one embodiment, preferred compounds of Formula IV are those of Formula IVc:

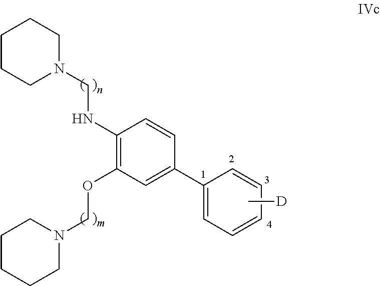

and pharmaceutically acceptable salts and solvates thereof,

wherein

n, m and D are as defined above with respect to Formula I and any of its embodiments.

In one embodiment, D is a group of formula

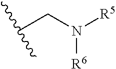

located at position 2 of the phenyl ring, wherein R⁵ and R⁶ are as defined above with respect to Formula I and any of its embodiments.

In one embodiment, D is a group of formula

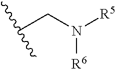

located at position 3 of the phenyl ring, wherein R⁵ and R⁶ are as defined above with respect to Formula I and any of its embodiments.

In one embodiment, D is a group of formula

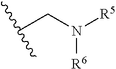

located at position 4 of the phenyl ring, wherein R⁵ and R⁶ are as defined above with respect to Formula I and any of its embodiments.

In one embodiment, preferred compounds of Formula IV are those of Formula IVd:

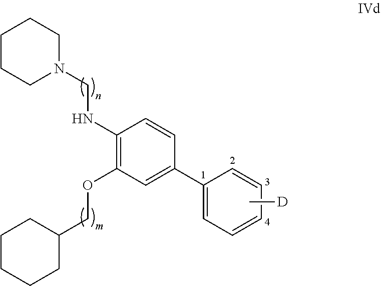

and pharmaceutically acceptable salts and solvates thereof,

wherein

n, m and D are as defined above with respect to Formula I and any of its embodiments.

In one embodiment, D is a group of formula

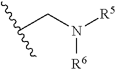

located at position 2 of the phenyl ring, wherein R⁵ and R⁶ are as defined above with respect to Formula I and any of its embodiments.

In one embodiment, D is a group of formula

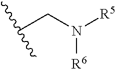

located at position 3 of the phenyl ring, wherein R⁵ and R⁶ are as defined above with respect to Formula I and any of its embodiments.

In one embodiment, D is a group of formula

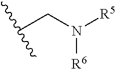

located at position 4 of the phenyl ring, wherein R⁵ and R⁶ are as defined above with respect to Formula I and any of its embodiments.

In one embodiment, preferred compounds of Formula IV are those of Formula IVe:

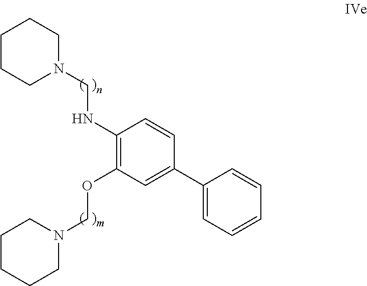

and pharmaceutically acceptable salts and solvates thereof,

wherein

n, m are as defined above with respect to Formula I and any of its embodiments.

In one embodiment, preferred compounds of Formula IV are those of Formula IVf:

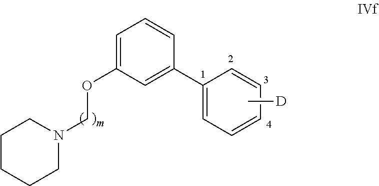

and pharmaceutically acceptable salts and solvates thereof,

wherein

m and D are as defined above with respect to Formula I and any of its embodiments.

Preferred compounds of Formula IVf are those wherein D is a group of formula

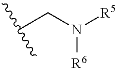

located at position 2 of the phenyl ring, wherein R⁵ and R⁶ are as defined above with respect to Formula I and any of its embodiments.

In one embodiment, preferred compounds of Formula IV are those of Formula IVg:

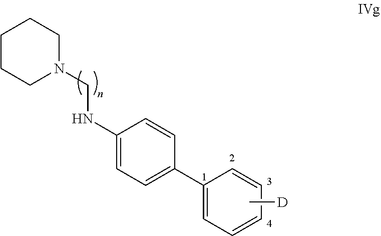

and pharmaceutically acceptable salts and solvates thereof,

wherein

n and D are as defined above with respect to Formula I and any of its embodiments.

Preferred compounds of Formula IVg are those wherein D is a group of formula

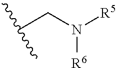

located at position 2 of the phenyl ring, wherein R⁵ and R⁶ are as defined above with respect to Formula I and any of its embodiments.

In one embodiment, preferred compounds of Formula I are those of Formula V:

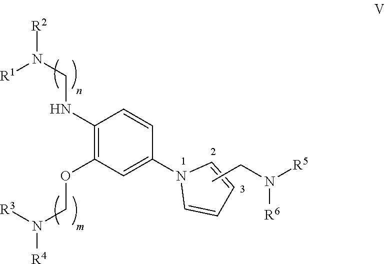

and pharmaceutically acceptable salts and solvates thereof,

wherein

R¹, R², R³, R⁴, n, m, R⁵ and R⁶ are as defined above with respect to Formula I and any of its embodiments.

Preferred compounds of formula V are those wherein the group of formula

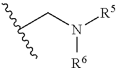

located at position 3 of the pyrrolyl group.

In one embodiment, preferred compounds of Formula V are those of Formula Va:

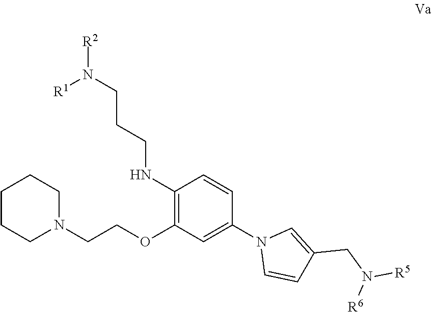

and pharmaceutically acceptable salts and solvates thereof,

wherein

R¹, R², R⁵ and R⁶ are as defined above with respect to Formula I and any of its embodiments.

Particularly preferred compounds of the invention are those listed in Table 1 hereafter:

TABLE 1

| Structure | Name |
|---|---|
| 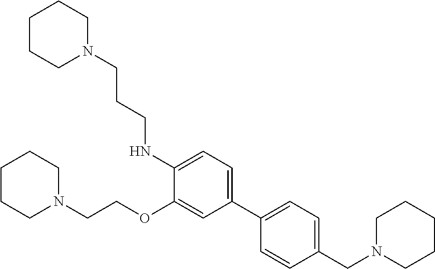 [SMILES: c1cc(-c2ccc(NCCCN3CCCCC3)c(OCCN3CCCCC3)c2)ccc1CN1CCCCC1] | 2-[2-(Piperidin-1-yl)ethoxy]-N-[3-(piperidin- 1-yl)propyl]-4-[4-(piperidin-1- ylmethyl)phenyl]aniline |
| 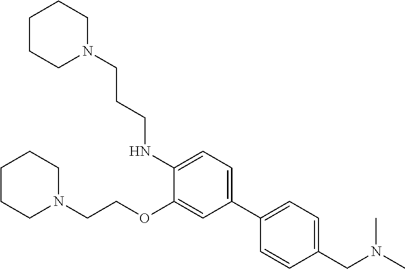 [SMILES: CN(C)Cc1ccc(-c2ccc(NCCCN3CCCCC3)c(OCCN3CCCCC3)c2)cc1] | 4-(4-[(Dimethylamino)methyl]phenyl)-2-[2- (piperidin-1-yl)ethoxy]-N-[3-(piperidin-1- yl)propyl]aniline |
| 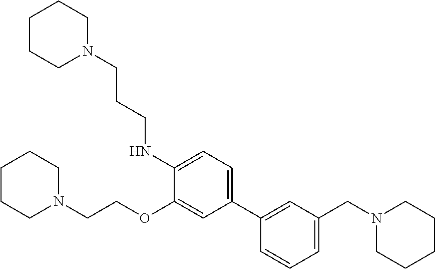 [SMILES: c1cc(CN2CCCCC2)cc(-c2ccc(NCCCN3CCCCC3)c(OCCN3CCCCC3)c2)c1] | 2-[2-(1-Piperidyl)ethoxy]-4-[3-(1- piperidylmethyl)phenyl]-N-[3-(1- piperidyl)propyl]aniline |
| 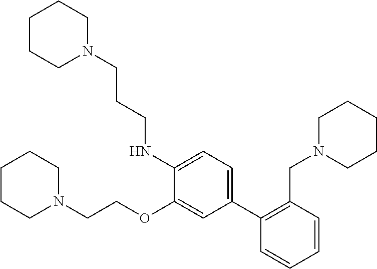 [SMILES: c1ccc(-c2ccc(NCCCN3CCCCC3)c(OCCN3CCCCC3)c2)c(CN2CCCCC2)c1] | 2-[2-(Piperidin-1-yl)ethoxy]-N-[3-(piperidin- 1-yl)propyl]-4-[2-(piperidin-1- ylmethyl)phenyl]aniline |
| 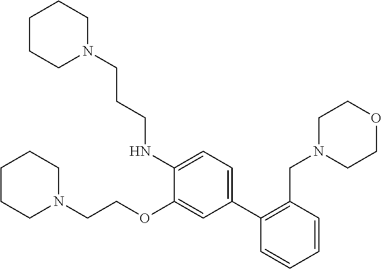 [SMILES: c1ccc(-c2ccc(NCCCN3CCCCC3)c(OCCN3CCCCC3)c2)c(CN2CCOCC2)c1] | 4-[2-(Morpholin-4-ylmethyl)phenyl]-2-[2- (piperidin-1-yl)ethoxy]-N-[3-(piperidin-1- yl)propyl]aniline |
| 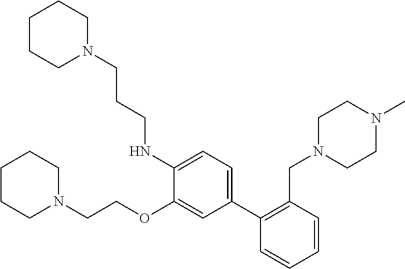 [SMILES: CN1CCN(Cc2ccccc2-c2ccc(NCCCN3CCCCC3)c(OCCN3CCCCC3)c2)CC1] | 4-(2-[(4-Methylpiperazin-1- yl)methyl]phenyl)-2-[2-(piperidin-1- yl)ethoxy]-N-[3-(piperidin-1-yl)propyl]aniline |
| 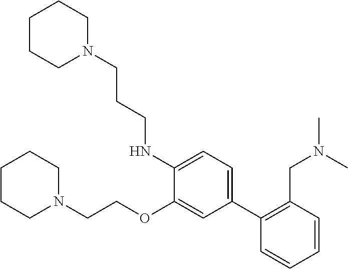 [SMILES: CN(C)Cc1ccccc1-c1ccc(NCCCN2CCCCC2)c(OCCN2CCCCC2)c1] | 4-(2-[(Dimethylamino)methyl]phenyl)-2-[2- (piperidin-1-yl)ethoxy]-N-[3-(piperidin-1- yl)propyl]aniline |
| 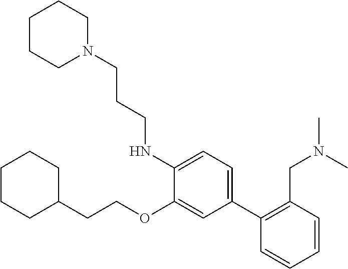 [SMILES: CN(C)Cc1ccccc1-c1ccc(NCCCN2CCCCC2)c(OCCC2CCCCC2)c1] | 3-(2-Cyclohexylethoxy)-2′- ((dimethylamino)methyl)-N-(3-(piperidin-1- yl)propyl)-[1,1′-biphenyl]-4-amine |
| 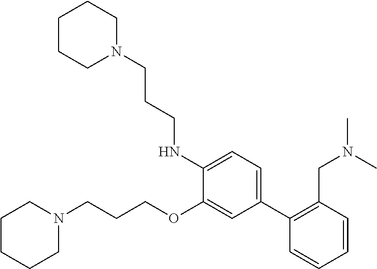 [SMILES: CN(C)Cc1ccccc1-c1ccc(NCCCN2CCCCC2)c(OCCCN2CCCCC2)c1] | 2′-((Dimethylamino)methyl)-3-(3-(piperidin- 1-yl)propoxy)-N-(3-(piperidin-1-yl)propyl)- [1,1′-biphenyl]-4-amine |
| 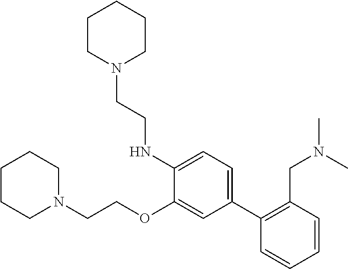 [SMILES: CN(C)Cc1ccccc1-c1ccc(NCCN2CCCCC2)c(OCCN2CCCCC2)c1] | 2′-((Dimethylamino)methyl)-3-(2-(piperidin- 1-yl)ethoxy)-N-(2-(piperidin-1-yl)ethyl)-[1,1′- biphenyl]-4-amine |
| 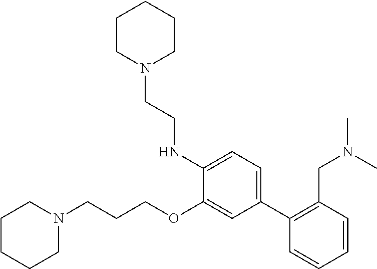 [SMILES: CN(C)Cc1ccccc1-c1ccc(NCCN2CCCCC2)c(OCCCN2CCCCC2)c1] | 2′-((Dimethylamino)methyl)-N-(2-(piperidin- 1-yl)ethyl)-3-(3-(piperidin-1-yl)propoxy)- [1,1′-biphenyl]-4-amine |
| 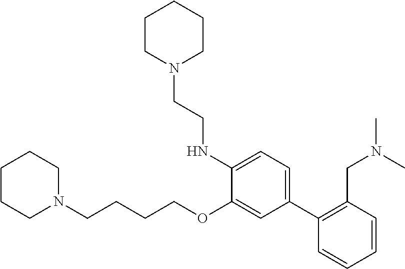 [SMILES: CN(C)Cc1ccccc1-c1ccc(NCCN2CCCCC2)c(OCCCCN2CCCCC2)c1] | 2′-((Dimethylamino)methyl)-3-(4-(piperidin- 1-yl)butoxy)-N-(2-(piperidin-1-yl)ethyl)-[1,1′- biphenyl]-4-amine |
| 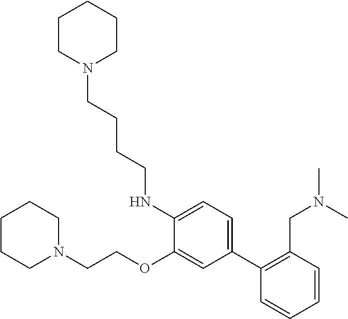 [SMILES: CN(C)Cc1ccccc1-c1ccc(NCCCCN2CCCCC2)c(OCCN2CCCCC2)c1] | 2′-((Dimethylamino)methyl)-N-(4-(piperidin- 1-yl)butyl)-3-(2-(piperidin-1-yl)ethoxy)-[1,1′- biphenyl]-4-amine |
| 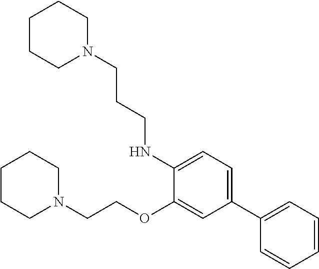 [SMILES: c1ccc(-c2ccc(NCCCN3CCCCC3)c(OCCN3CCCCC3)c2)cc1] | 3-(2-(Piperidin-1-yl)ethoxy)-N-(3-(piperidin- 1-yl)propyl)-[1,1′-biphenyl]-4-amine |
| 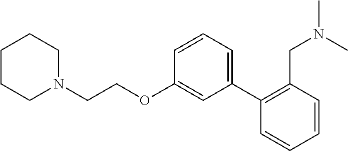 [SMILES: CN(C)Cc1ccccc1-c1cccc(OCCN2CCCCC2)c1] | N,N-Dimethyl-1-(3′-(2-(piperidin-1- yl)ethoxy)-[1,1′-biphenyl]-2-yl)methanamine |
| 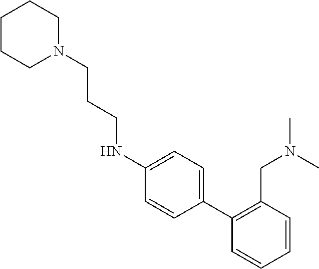 [SMILES: CN(C)Cc1ccccc1-c1ccc(NCCCN2CCCCC2)cc1] | 2′-((Dimethylamino)methyl)-N-(3-(piperidin- 1-yl)propyl)-[1,1′-biphenyl]-4-amine |
| 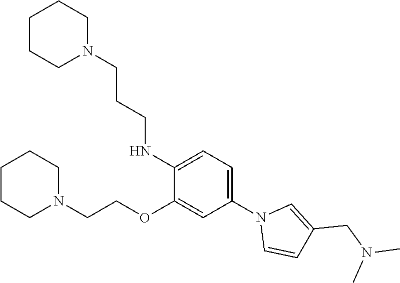 [SMILES: CN(C)Cc1ccn(-c2ccc(NCCCN3CCCCC3)c(OCCN3CCCCC3)c2)c1] | 4-(3-[(Dimethylamino)methyl]-1H-pyrrol-1- yl)-2-[2-(piperidin-1-yl)ethoxy]-N-[3- (piperidin-1-yl)propyl]aniline |
| 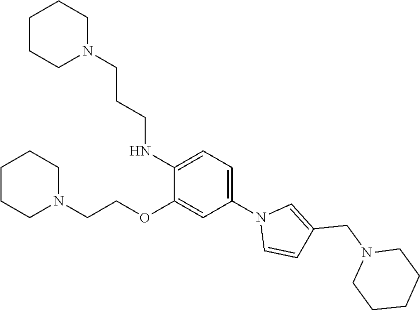 [SMILES: c1cn(-c2ccc(NCCCN3CCCCC3)c(OCCN3CCCCC3)c2)cc1CN1CCCCC1] | 2-[2-(Piperidin-1-yl)ethoxy]-N-[3-(piperidin- 1-yl)propyl]-4-[3-(piperidin-1-ylmethyl)-1H- pyrrol-1-yl]aniline |
| 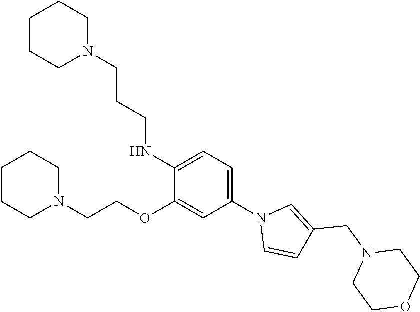 [SMILES: c1cn(-c2ccc(NCCCN3CCCCC3)c(OCCN3CCCCC3)c2)cc1CN1CCOCC1] | 4-[3-(Morpholin-4-ylmethyl)-1H-pyrrol-1- yl]-2-[2-(piperidin-1-yl)ethoxy]-N-[3- (piperidin-1-yl)propyl]aniline |
| 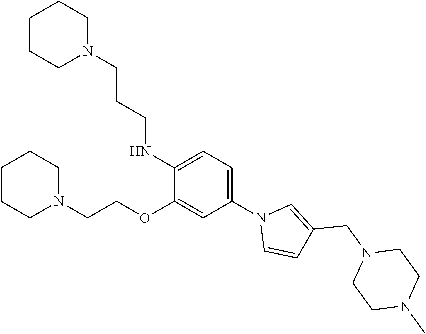 [SMILES: CN1CCN(Cc2ccn(-c3ccc(NCCCN4CCCCC4)c(OCCN4CCCCC4)c3)c2)CC1] | 4-(3-[(4-Methylpiperazin-1-yl)methyl]-1H- pyrrol-1-yl)-2-[2-(piperidin-1-yl)ethoxy]-N- [3(piperidin-1-yl)propyl]aniline |
| 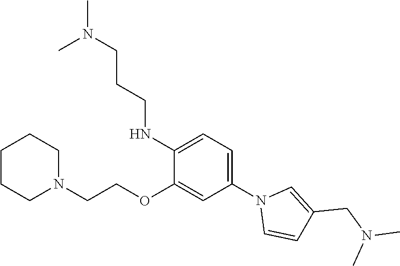 [SMILES: CN(C)CCCNc1ccc(-n2ccc(CN(C)C)c2)cc1OCCN1CCCCC1] | 4-(3-[(dimethylamino)methyl]-1H-pyrrol-1- yl)-N-[3-(dimethylamino)propyl]-2-[2- (piperidin-1-yl)ethoxy]aniline |
| 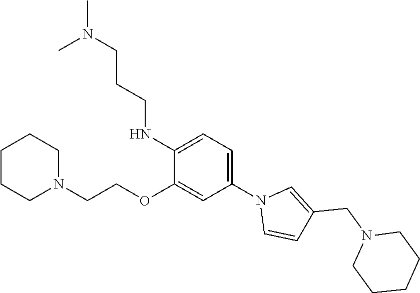 [SMILES: CN(C)CCCNc1ccc(-n2ccc(CN3CCCCC3)c2)cc1OCCN1CCCCC1] | N-[3-(Dimethylamino)propyl]-2-[2- (piperidin-1-yl)ethoxy]-4-[3-(piperidin-1- ylmethyl)-1H-pyrrol-1-yl]aniline |
| 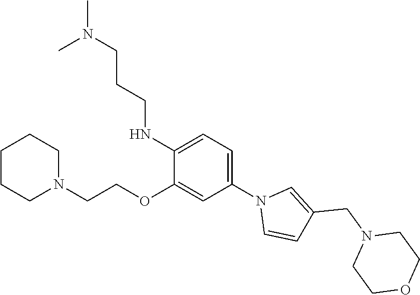 [SMILES: CN(C)CCCNc1ccc(-n2ccc(CN3CCOCC3)c2)cc1OCCN1CCCCC1] | N-[3-(Dimethylamino)propyl]-4-[3- (morpholine-4-ylmethyl)-1H-pyrrol-1-yl]-2- [2-(piperidin-1-yl)ethoxy]aniline |
| 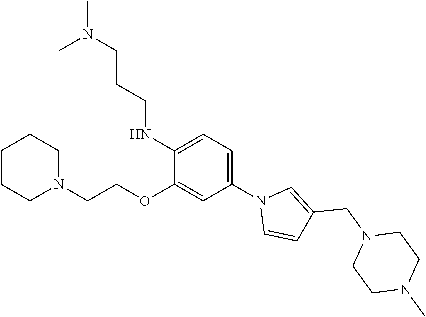 [SMILES: CN(C)CCCNc1ccc(-n2ccc(CN3CCN(C)CC3)c2)cc1OCCN1CCCCC1] | N-[3-(Dimethylamino)propyl]-4-(3-[(4- methylpiperazin-1-yl)methyl]-1H-pyrrol-1- yl}-2-[2-(piperidin-1-yl)ethoxy]aniline |

The compounds of the invention can be prepared by different ways with reactions known by the person skilled in the art. Reaction schemes as described in the example section illustrate by way of example different possible approaches.

The compounds of the invention are indeed modulators, preferably inhibitors of Aβ peptides secretion. They further have the advantage of being able to promote αCTFs and AICD expression. The invention thus also provides the use of the compounds of the invention or pharmaceutically acceptable salts, or solvates thereof as inhibitors of Aβ peptides secretion and promotors of αCTFs and AICD expression.

Accordingly, in a particularly preferred embodiment, the invention relates to the use of compounds of Formula I and subformulae in particular those of Table 1 above, or pharmaceutically acceptable salts and solvates thereof, as inhibitors of Aβ peptides production and/or stabilizers of αCTFs and AICD expression.

### APPLICATIONS

Unexpectedly, the inventors have discovered that the compounds of formula I according to the present invention may be used to rectify the metabolism of amyloid protein precursor (APP) of at least two of the four following essential points, preferably points 3) and 4):
1) increasing the carboxy-terminal fragments of APP (APP-CTFs) which all in common possess the last 50 aminoacids of APP, and especially those having potential physiological activities, such as the α-stubs (APP-CTFs a) and the AICD (APP intra cellular domain) with neurotrophic properties,
2) having an indirect β-secretase activity by reduction the production of the soluble form sAPPβ but not modifying the production of sAPPα,
3) decreasing the production of the neurotoxic by-products of APP, i.e. β-amyloid (Aβ) peptides, especially in their form 1-40 and 1-42,
4) without modifying the APP expression and in absence of neurotoxicity.

The compounds of the invention are therefore useful in the prevention and/or treatment of diseases involving formation of amyloid plaques and/or where a dysfunction of the APP metabolism occurs.

The invention thus also relates to a compound of the invention or a pharmaceutically acceptable salt or solvate thereof for use in treating and/or preventing a disease or disorder involving formation of amyloid plaques and/or where a dysfunction of the APP metabolism occurs. Or in other terms, the invention also relates to a method of treating and/or preventing a disease or disorder involving formation of amyloid plaques and/or where a dysfunction of the APP metabolism occurs, comprising the administration of a therapeutically effective amount of a compound or pharmaceutically acceptable salt or solvate of the invention, to a patient in need thereof. Preferably the patient is a warm-blooded animal, more preferably a human.

Diseases involving formation of amyloid plaques and/or where a dysfunction of the APP metabolism occurs within the meaning of the present invention include, but are not limited to Alzheimer's disease (AD), Lewy body disease, Down syndrome, amyloid angiopathy, Parkinson's disease (PD), prion diseases, in particular Creutzfeldt-Jakob Disease (CJD), amyotrophic lateral sclerosis (ALS), and frontotemporal degeneration. In a particular preferred embodiment, the disease involving formation of amyloid plaques and/or where a dysfunction of the APP metabolism occurs is Alzheimer's disease.

The invention further provides the use of a compound of the invention or a pharmaceutically acceptable salt or solvates thereof for the manufacture of a medicament for use in treating and/or preventing a disease or disorder involving formation of amyloid plaques and/or where a dysfunction of the APP metabolism occurs. Preferably the patient is a warm-blooded animal, more preferably a human. The diseases or disorders involving formation of amyloid plaques and/or where a dysfunction of the APP metabolism occurs are preferably those defined above.

According to a further feature of the present invention, there is provided a compound of the invention or a pharmaceutically acceptable salt or solvate for use in modulating, preferably inhibiting Aβ peptides production and/or stabilizing αCTFs and AICD expression in a patient in need of such treatment, comprising administering to said patient an effective amount of a compound of the present invention, or a pharmaceutically acceptable salt or solvate thereof. In other terms, the invention also provides a method for modulating, preferably inhibiting Aβ peptides production and/or stabilizing αCTFs and AICD expression, in a patient in need of such treatment, which comprises administering to said patient an effective amount of a compound of the present invention, or a pharmaceutically acceptable salt or solvate thereof. Preferably, the patient is a warm blooded animal, and even more preferably a human.

According to one embodiment, the compounds of the invention, their pharmaceutical acceptable salts or solvates may be administered as part of a combination therapy. Thus, are included within the scope of the present invention embodiments comprising co-administration of, and compositions and medicaments which contain, in addition to a compound of the present invention, a pharmaceutically acceptable salt or solvate thereof as active ingredient, additional therapeutic agents and/or active ingredients. Such multiple drug regimens, often referred to as combination therapy, may be used in the treatment and/or prevention of any disease or disorder involving formation of amyloid plaques and/or where a dysfunction of the APP metabolism occurs, particularly those defined above.

Thus, the methods of treatment and pharmaceutical compositions of the present invention may employ the compounds of the invention or their pharmaceutical acceptable salts or solvates thereof in the form of monotherapy, but said methods and compositions may also be used in the form of multiple therapy in which one or more compounds of Formula I or their pharmaceutically acceptable salts or solvates are co-administered in combination with one or more other therapeutic agents.

The invention also provides pharmaceutical compositions comprising a compound of the invention or a pharmaceutically acceptable salt or solvate thereof and at least one pharmaceutically acceptable carrier, diluent, excipient and/or adjuvant. As indicated above, the invention also covers pharmaceutical compositions which contain, in addition to a compound of the present invention, a pharmaceutically acceptable salt or solvate thereof as active ingredient, additional therapeutic agents and/or active ingredients.

Another object of this invention is a medicament comprising at least one compound of the invention, or a pharmaceutically acceptable salt or solvate thereof, as active ingredient.

Generally, for pharmaceutical use, the compounds of the invention may be formulated as a pharmaceutical preparation comprising at least one compound of the invention and at least one pharmaceutically acceptable carrier, diluent, excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), cerebral administration, sublingual administration, aerosol administration, for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

### Definitions

The definitions and explanations below are for the terms as used throughout the entire application, including both the specification and the claims.

Unless otherwise stated any reference to compounds of the invention herein, means the compounds as such as well as their pharmaceutically acceptable salts and solvates.

When describing the compounds of the invention, the terms used are to be construed in accordance with the following definitions, unless indicated otherwise.

The term “halo” or “halogen” refers to the atoms of the group 17 of the periodic table (halogens) and includes in particular fluorine, chlorine, bromine and iodine atom.

The term “alkyl” by itself or as part of another substituent refers to a hydrocarbyl radical of Formula CₙH₂ₙ₊₁ wherein n is a number greater than or equal to 1.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

The term “cycloalkyl” as used herein is a monovalent, saturated, or unsaturated monocyclic or bicyclic hydrocarbyl group. Cycloalkyl groups may comprise 3 or more carbon atoms in the ring and generally, according to this invention comprise from 3 to 10, more preferably from 3 to 8 carbon atoms still more preferably from 3 to 6 carbon atoms. Examples of cycloalkyl groups include but are not limited to cyclopropyl, cyclobutyl, cyclopentyl, and cyclohexyl.

The term “heteroatom” as used herein refers to any atom that is not carbon or hydrogen. Non-limiting examples of such heteroatoms include nitrogen, oxygen, sulfur, and phosphorus. Preferred heteroatoms are nitrogen and oxygen.

The terms “heterocyclyl”, “heterocycloalkyl” or “heterocyclo” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen, oxygen and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. Examples of heterocyclyl groups include but are not limited to aziridinyl, azetidinyl, pyrrolidinyl, piperidinyl, azepanyl, piperazinyl, morpholinyl, homopiperazinyl. Preferred heterocyclyl group according to the invention are piperidinyl, piperazinyl, morpholinyl and homopiperazinyl. More preferred heterocyclyl group according to the invention are piperidinyl, piperazinyl, and morpholinyl.

The term “aryl” as used herein refers to a polyunsaturated, aromatic hydrocarbyl group having a single ring (i.e. phenyl) or multiple aromatic rings fused together (e.g. naphtyl), typically containing 5 to 12 atoms; preferably 6 to 10, wherein at least one ring is aromatic. Examples of aryl groups include but are not limited to phenyl, naphtyl, anthracyl. Preferred aryl group according to the invention is phenyl.

According to the present invention, carbon atoms of 5- or 6-membered aryl group C as defined in Formula I and any of its embodiments are numbered from 1 to 5 or from 1 to 6, the carbon in position 1 being the carbon of group C linked to the central phenyl group of Formula I.

The term “heteroaryl” as used herein by itself or as part of another group refers but is not limited to 5 to 12 carbon-atom aromatic rings or ring systems containing 1 to 2 rings which are fused together, typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Examples of heteroaryl groups include but are not limited to pyridyl, pyrazinyl, pyrimidinyl, pyridazinyl, triazinyl, quinolinyl, quinoxalinyl, quinazolinyl, furanyl, benzofuranyl, pyrrolyl, indolyl, thiophenyl, benzothiophenyl, imidazolyl, benzimidazolyl, pyrazolyl, indazolyl, oxazolyl, benzoxazolyl, isoxazolyl, benzisoxazolyl, thiazolyl, and benzothiazolyl. Preferred heteroaryl group according to the invention is pyrrolyl.

According to the present invention, atoms of 5- or 6-membered heteroaryl group C as defined in Formula I and any of its embodiments are numbered from 1 to 5 or from 1 to 6, the atom in position 1 being the atom of group C linked to the central phenyl group of Formula I.

The compounds of the invention containing a basic functional group may be in the form of pharmaceutically acceptable salts. Pharmaceutically acceptable salts of the compounds of the invention containing one or more basic functional groups include in particular the acid addition salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, cinnamate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Pharmaceutically acceptable salts of compounds of Formula I and subformulae may for example be prepared as follows:

(i) reacting the compound of Formula I or any of its subformulae with the desired acid; or

(ii) converting one salt of the compound of Formula I or any of its subformulae to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The term “solvate” is used herein to describe a molecular complex comprising the compound of the invention and one or more pharmaceutically acceptable solvent molecules, for example, ethanol. The term “hydrate” is employed when said solvent is water.

The compounds of the invention include compounds of the invention as hereinbefore defined, including all polymorphs and crystal habits thereof, prodrugs and isomers thereof (including optical, geometric and tautomeric isomers) and isotopically-labeled compounds of the invention.

In addition, although generally, with respect to the salts of the compounds of the invention, pharmaceutically acceptable salts are preferred, it should be noted that the invention in its broadest sense also includes non-pharmaceutically acceptable salts, which may for example be used in the isolation and/or purification of the compounds of the invention.

The term “patient” refers to a warm-blooded animal, more preferably a human, who/which is awaiting or receiving medical care or is or will be the object of a medical procedure.

The term “human” refers to subjects of both genders and at any stage of development (i.e. neonate, infant, juvenile, adolescent, adult). In one embodiment, the human is an adolescent or adult, preferably an adult.

The terms “treat”, “treating” and “treatment”, as used herein, are meant to include alleviating or abrogating a condition or disease and/or its attendant symptoms.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “therapeutically effective amount” (or more simply an “effective amount”) as used herein means the amount of active agent or active ingredient which is sufficient to achieve the desired therapeutic or prophylactic effect in the individual to which it is administered.

The term “administration”, or a variant thereof (e.g., “administering”), means providing the active agent or active ingredient, alone or as part of a pharmaceutically acceptable composition, to the patient in whom/which the condition, symptom, or disease is to be treated or prevented.

By “pharmaceutically acceptable” is meant that the ingredients of a pharmaceutical composition are compatible with each other and not deleterious to the patient thereof.

The term “agonist” as used herein means a ligand that activates an intracellular response when it binds to a receptor.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, and liposomes.

The present invention will be better understood with reference to the following examples and figures. These examples are intended to representative of specific embodiments of the invention, and are not intended as limiting the scope of the invention.

### FIGURES

FIG. 1: Effect of compounds of the invention on APP processing: αCTF (A) and AICD (B) stability.

FIG. 2: Effect of compounds 30 and 31 on APP metabolism in SY5Y-APP695WT cells.

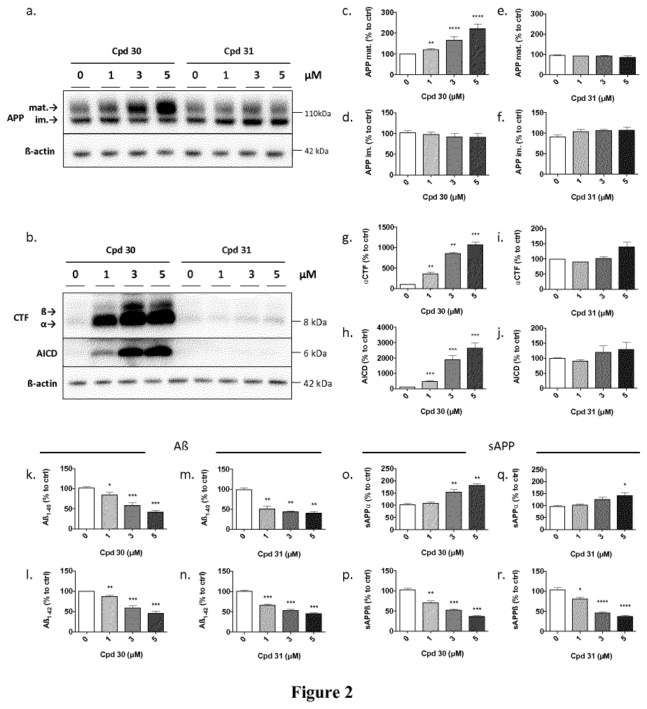

FIG. 3: Effect of compounds 30 and 31 on the autophagy flux in SY5Y-APP^{695WT} cells.

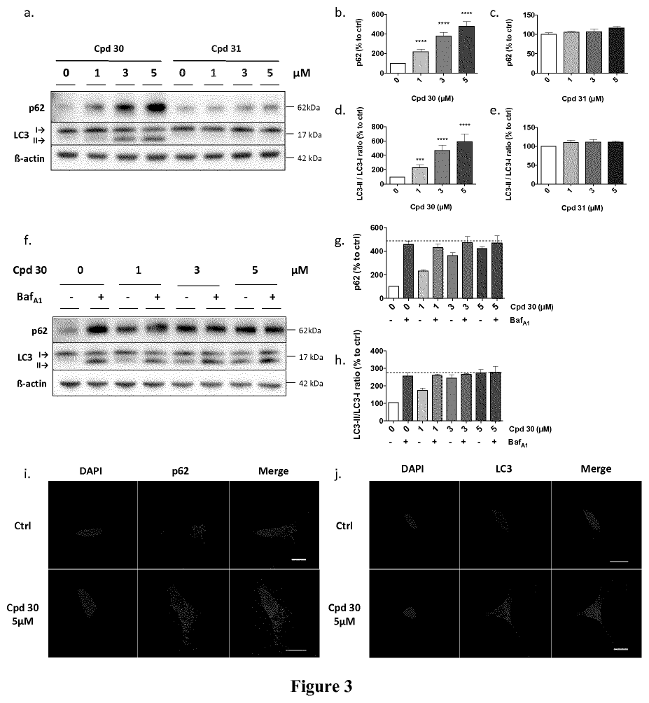

FIG. 4: Effect of compounds 30 and 31 on ThyTau22 mice behavior (Y-Maze).

FIG. 5: Biochemical analysis of Tau expression and phosphorylation by Western-blot for compound 31.

FIG. 6: Amount of Tau phosphorylation for compound 31 in a) the cortex and b) the hippocampus.

### EXAMPLES

### Chemistry Examples

All temperatures are expressed in ° C. and all reactions were carried out at room temperature (RT) unless otherwise stated.

The reaction monitoring was performed by thin layer chromatography (TLC) on Macherey-Nagel Alugram® Sil 60/UV254 (thickness 0.2 mm). TLC were revealed by UV (2=254 nm) and/or the appropriate stain.

Purification of the compounds was carried out by column chromatography (either flash or manual). Manual chromatography was performed using Macherey-Nagel silica gel (0.04-0.063 mm of particule size). Flash chromatography was performed on a Reveleris® Flash Chromatography System using Macherey-Nagel Chromabond flash RS columns.

NMR spectra were recorded on a Bruker DRX 300 spectrometer (operating at 300 MHz for ¹H and 75 MHz for ¹³C). Chemical shifts are expressed in ppm relative to tetramethylsilane (TMS) or to residual proton signal in deuterated solvents. Chemical shifts are reported as position (δ in ppm), multiplicity (s=singulet, d=doublet, t=triplet, q=quartet, m=multiplet, br=broad), coupling constant (J in Hz) and relative integral. The attributions of protons and carbons were achieved by analysis of 1D and 2D experiments (¹H, ¹³C, COSY, HQC and HMBC).

LC-MS were performed on a Varian triple quadrupole 1200W mass spectrometer equipped with a non-polar C18 TSK-gel Super ODS (4.6×50 mm) column, using electrospray ionisation and a UV detector (diode array). Elution was performed at a flow rate of 2 mL/min with water-formic acid (pH=3.8) as eluent A and ACN-formic acid (pH=3.8) as eluent B, employing a 0.25 min plateau with 0% B and a linear gradient from 0% B to 98% B in 3.25 min, followed by a 0.5 min plateau with 98% B. Then, column re-equilibration was performed for 1 min.

HRMS were recorded on a High Resolution Mass Spectrometer (HRMS) Thermo Scientific™ Exactive™. Analysed compounds were dissolved in methanol and directly injected in the ionisation source ESI, in positive or negative mode according to the analyzed compound, and recorded for one minute. The Xcalibur software was used to determine the elemental composition of main pics of the spectrum.

The purity of final compounds was determined by high pressure liquid chromatography (HPLC) using two columns: C18 Interchrom UPTISPHERE and C4 Interchrom UPTISPHERE. The HPLC analysis was carried out on a Shimadzu LC-2010AHT system equipped with a UV detector set at 254 and 215 nm. The compounds were dissolved in 100 μL of buffer B and 900 μL of buffer A. The eluent system used was: buffer A (H₂O/TFA, 100:0.1) and buffer B (ACN/H₂O/TFA, 80:20:0.1). Retention times (tr) were obtained at a flow rate of 0.2 mL/min for 37 min using a gradient form 100% of buffer A over 1 min, to 100% buffer B over the next 30 min, to 100% of buffer A over 1 min and 100% of buffer A over 1 min.

The melting point analyses were performed on Barnstead Electrothermel Melting Point Series IA9200 and were not corrected.

All final compounds were transformed into their hydrochloride salt forms (before testing) using the following procedure: the compound was dissolved in MeOH and 2N HCl_{aq} was added dropwise until pH1. The solvent was evaporated and the compound was freeze-dried.

Solvents, reagents and starting materials were purchased from well known chemical suppliers such as for example Sigma Aldrich, Acros Organics, Fluorochem, Eurisotop, VWR International, Sopachem and Polymer labs and the following abbreviations are used:

ACN: Acetonitrile,

DCM: Dichloromethane,

DMF: N,N-dimethylformamide,

DMSO: Dimethylsulfoxyde,

eq: Equivalent,

EtOH: Ethanol,

LCMS: Liquid chromatography-mass spectrometry,

MeOH: Methanol,

Mp: Melting point,

MW: Molecular weight,

PE: Petroleum ether,

rt: Room temperature,

TLC: Thin layer chromatography.

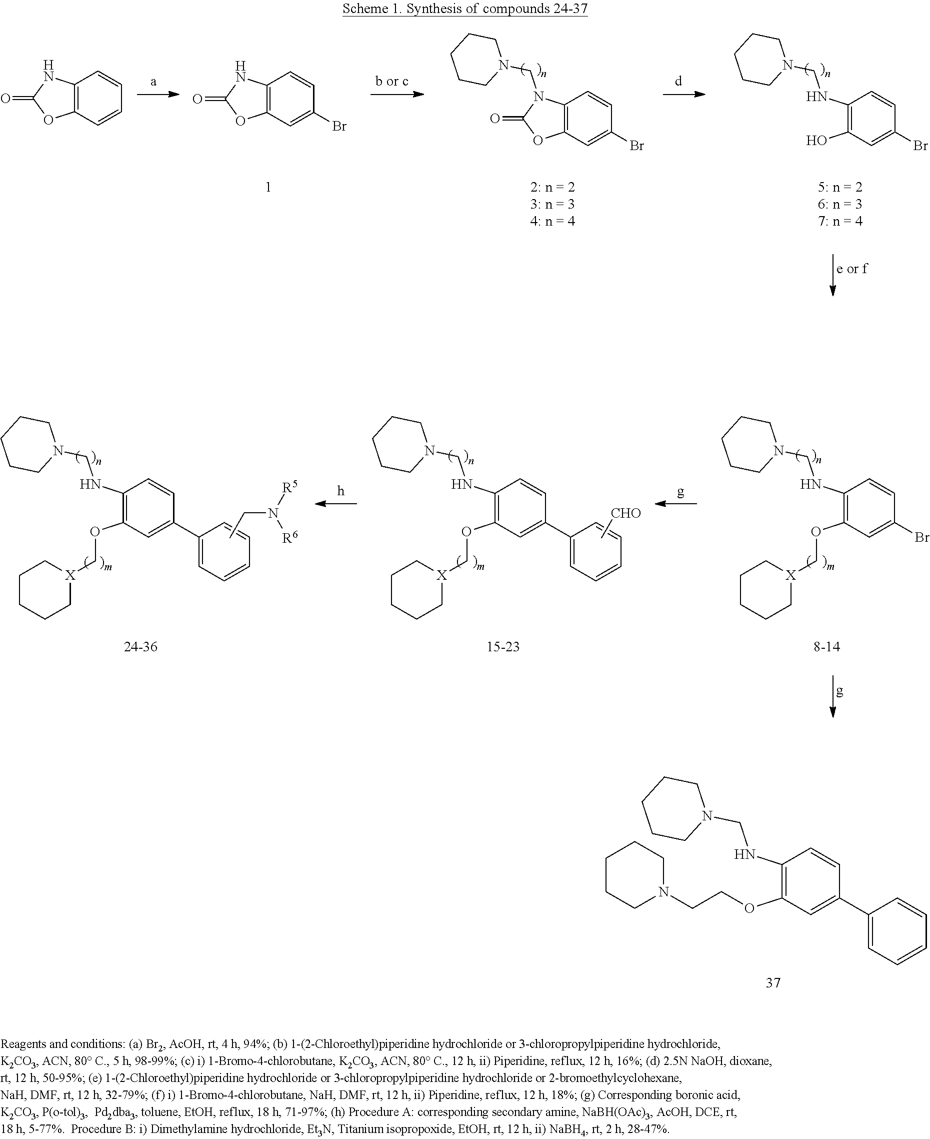

### 6-Bromo-3H-1,3-benzoxazol-2-one (1)

3H-1,3-Benzoxazol-2-one (5.00 g, 37.00 mmol) was dissolved in acetic acid (50 mL) and bromine (1.9 mL, 37.0 mmol) was added dropwise. The reaction mixture was stirred at 20° C. for 4 h. The reaction mixture was poured onto ice and the precipitate was collected by filtration, washed with water and air-dried to give 1 as a pink powder (7.48 g, 94%). Mp 191.6° C. ¹H NMR (300 MHz), δ (ppm, DMSO-d₆): 11.81 (s, 1H), 7.57 (dd, J=1.9 Hz, J=0.3 Hz, 1H), 7.30 (dd, J=8.3 Hz, J=1.9 Hz, 1H), 7.04 (dd, J=8.3 Hz, J=0.3 Hz, 1H). ¹³C NMR (75 MHz), δ (ppm, DMSO-d₆): 154.5, 144.5, 130.3, 126.9, 113.5, 113.2, 111.7. LCMS m/z calc for [M−H]⁺: 211.9, 213.9, found: 211.8, 213.8.

### General Procedure for the Synthesis of Compounds 2 and 3

6-bromo-3H-1,3-benzoxazol-2-one 1 (5.00 g, 23.36 mmol) was suspended in ACN (150 mL) and K₂CO₃ (9.69 g, 70.09 mmol) was added. The reaction mixture was stirred at 80° C. for 30 min. 1-(2-Chloroethyl)piperidine hydrochloride (4.30 g, 23.36 mmol) or 3-chloropropylpiperidine hydrochloride (5.5 g, 28 mmol) was added and the reaction mixture was stirred at 80° C. for another 12 h. The inorganics were removed by filtration and the solvent was evaporated. The residue was purified by flash chromatography (DCM/MeOH(NH₃), 9.8:0.2 (v/v)) to afford compounds 2 and 3.

### 6-Bromo-3-[2-(piperidin-1-yl)ethyl]-1,3-benzoxazol-2-one (2)

Compound 2 was obtained as a beige solid (7.58 g, 99%). Mp 85° C. ¹H NMR (300 MHz), δ (ppm, CDCl₃): 7.36 (d, J=1.8 Hz, 1H), 7.31 (dd, J=8.3, 1.8 Hz, 1H), 6.92 (d, J=8.3 Hz, 1H), 3.90 (t, J=6.6 Hz, 2H), 2.64 (t, J=6.6 Hz, 2H), 2.51-2.38 (m, 4H), 1.60-1.35 (m, 6H). ¹³C NMR (75 MHz), δ (ppm, CDCl₃): 154.1, 143.1, 130.7, 126.6, 114.4, 113.5, 109.9, 56.0, 54.7, 40.3, 26.0, 24.2. LCMS m/z calc for [M+H]⁺: 325.1, 327.1 found: 325.1, 327.1.

### 6-Bromo-3-[3-(piperidin-1-yl)propyl]-1,3-benzoxazol-2-one (3)

Compound 3 was obtained as a colorless oil (7.76 g, 98%). ¹H NMR (300 MHz), δ (ppm, CDCl₃): 7.36 (d, J=1.7 Hz, 1H), 7.30 (dd, J=8.3 Hz, J=1.9 Hz, 1H), 6.98 (d, J=8.3 Hz, 1H), 3.88 (t, J=6.7 Hz, 2H); 2.35-2.28 (m, 6H), 1.98-1.89 (m, 2H), 1.60-1.51 (m, 4H), 1.46-1.38 (m, 2H). ¹³C NMR (75 MHz), δ (ppm, CDCl₃): 154.1, 143.1, 130.8, 126.6, 114.4, 113.5, 109.7, 55.5, 54.5, 40.6, 25.9, 24.8, 24.4. LCMS m/z calc for [M+H]⁺: 338.1, 340.1, found: 338.9, 340.8.

### 6-bromo-3-[4-(piperidin-1-yl)butyl]-1,3-benzoxazol-2-one (4)

To a solution of 1-bromo-4-chlorobutane (2.17 mL, 18.7 mmol), K₂CO₃ (3.87 g, 28.04 mmol) in ACN (1 mL) was added dropwise at a solution of 6-bromo-3H-1,3-benzoxazol-2-one 1 in ACN (3 mL). The mixture was heated at 80° C. for 12 h. The inorganics were removed by filtration and the solvent was evaporated. The residue was purified by column chromatography (cyclohexane/ethyl acetate), 9:1 (v:v)) to afford 6-bromo-3-(4-chlorobutyl)-1,3-benzoxazol-2-one as a white solid (1.2 g). Mp 62° C. ¹H NMR (300 MHz), δ (ppm, CDCl₃): 7.39 (d, J=1.7 Hz, 1H), 7.34 (dd, J=8.3, 1.8 Hz, 1H), 6.88 (d, J=8.3 Hz, 1H), 3.87 (t, J=6.8 Hz, 2H), 3.60 (t, J=6.1 Hz, 2H), 2.27-1.74 (m, 4H). ¹³C NMR (75 MHz), δ (ppm, CDCl₃): 154.1, 143.1, 130.2, 126.9, 114.8, 113.8, 109.2, 77.5, 77.0, 76.6, 44.0, 41.6, 29.2, 25.0. LCMS m/z calc for [M+ACN+H]⁺: 345.6, 347.6, found: 345.1, 347.1.

6-Bromo-3-(4-chlorobutyl)-1,3-benzoxazol-2-one (1.2 g, 3.94 mmol) was dissolved in piperidine (19 mL) and refluxed for 12 h. The mixture was concentrated in vacuo and the residue was dissolved in 1N NaOH (20 mL) and extracted with DCM (3×60 mL). The combined organic layers were dried over MgSO₄, filtrated and concentrated in vacuo. The residue was purified by column chromatography (DCM/MeOH(NH₃), 9.5:0.5 (v:v)) to afford 4 as a beige powder (1.07 g, 32% over two steps). Mp 100° C. ¹H NMR (300 MHz), δ (ppm, CDCl₃): 7.37 (d, J=1.7 Hz, 1H), 7.32 (dd, J=8.3, 1.8 Hz, 1H), 6.88 (d, J=8.3 Hz, 1H), 3.83 (t, J=7.2 Hz, 2H), 2.38-2.27 (m, 6H), 1.86-1.70 (m, 2H), 1.66-1.42 (m, 8H). ¹³C NMR (75 MHz), δ (ppm, CDCl₃): 154.0, 143.1, 130.4, 126.7, 114.6, 113.6, 109.4, 58.5, 58.4, 54.6, 42.3, 42.2, 30.0, 25.8, 24.4, 23.9. LCMS m/z calc for [M+H]⁺: 353.1, 355.1, found: 353.1, 355.1.

### General Procedure for the Synthesis of Compounds 5-7 is Exemplified by the Protocol Used for the Synthesis of 5

To a solution of 6-bromo-3-[2-(piperidin-1-yl)ethyl]-1,3-benzoxazol-2-one (2) in dioxane (19 mL), aqueous 2.5N NaOH (98.4 mL, 246 mmol) was added. The mixture was stirred at 20° C. for 12 h. After cooling to 0° C., a 1N HCl solution was added to reach pH 8. After 30 min of stirring, the mixture was extracted with DCM (3×100 mL). The combined organic layers were dried over MgSO₄ and evaporated to afford 5-bromo-2-((2-(piperidin-1-yl)ethyl)amino)phenol 5 as a beige solid (3.1 g, 84%) that was used without further purification. Mp 127° C. ¹H NMR (300 MHz), δ (ppm, CDCl₃): 6.95 (d, J=2.1 Hz, 1H), 6.87 (dd, J=8.3, 2.1 Hz, 1H), 6.66 (d, J=8.3 Hz, 1H), 3.25-3.15 (t, J=5.7 Hz, 2H), 2.60-2.31 (m, 6H), 1.75-1.64 (m, 4H), 1.56-1.45 (m, 2H). ¹³C NMR (75 MHz), δ (ppm, CDCl₃): 151.1, 135.8, 122.7, 120.9, 119.8, 113.8, 57.3, 54.3, 44.7, 25.0, 23.9. LCMS m/z calc for [M+H]⁺: 299.1, 301.1, found: 299.1, 301.1.

### 5-Bromo-2-([3-(piperidin-1-yl)propyl]amino)phenol (6)

Compound 6 was obtained as a brown oil (2.16 g, 95%). ¹H NMR (300 MHz), δ (ppm, MeOD): 6.82-6.78 (m, 2H), 6.49 (d, J=8.1 Hz, 1H), 3.15 (t, J=6.7 Hz, 2H), 2.57-2.52 (m, 6H), 1.89-1.80 (m, 2H), 1.69-1.62 (m, 4H), 1.54-1.50 (m, 2H). ¹³C NMR (75 MHz), δ (ppm, MeOD): 145.8, 136.7, 122.1, 115.9, 111.5, 107.6, 56.7, 53.9, 41.8, 25.0, 24.7, 23.4. LCMS m/z calc for [M+H]⁺: 313.1, 315.1, found: 312.9, 314.9.

### 5-bromo-2-[(4-(piperidin-1-yl)butyl)amino]phenol (7)

Compound 7 was obtained as a beige solid (246 mg, 50%). Mp 130° C. ¹H NMR (300 MHz), δ (ppm, DMSO-d₆): 6.79-6.73 (m, 2H), 6.40 (d, J=9.0 Hz, 1H), 3.02 (t, J=6.5 Hz, 2H), 2.39-2.22 (m, 6H), 1.59-1.27 (m, 12H). ¹³C NMR (75 MHz), δ (ppm, DMSO-d₆): 145.2, 137.1, 121.9, 115.7, 110.6, 105.4, 58.0, 53.9, 42.5, 40.3, 40.1, 39.8, 39.5, 39.2, 39, 38.7, 26.3, 25.4, 24.0, 23.8. LCMS m/z calc for [M+H]⁺: 327.1, 329.1 found: 327.2, 329.2.

### General Procedure for the Synthesis of Compounds 8, 9, 11-14 is Exemplified by the Protocol Used for the Synthesis of 8

To a solution of 5-bromo-2-([2-(piperidin-1-yl)ethyl]amino)phenol (5) (550 mg, 1.84 mmol) in DMF (23 mL), at 0° C., NaH (147 mg, 3.68 mmol) was added. After stirring the mixture for 30 min, 1-(2-chloroethyl)piperidine hydrochloride (338 mg, 1.84 mmol) was added at 0° C. The reaction mixture was stirred for another 12 h at rt. The mixture was diluted with 20 mL of water and extracted with DCM (3×60 mL). The combined organic layers were dried over MgSO₄ and evaporated. The residue was purified by column chromatography (DCM/MeOH(NH₃), 9.5:0.5 (v:v)) to afford 4-bromo-2-(2-(piperidin-1-yl)ethoxy)-N-(2-(piperidin-1-yl)ethyl)aniline 8 as a brown oil (510 mg, 78%). ¹H NMR (300 MHz), δ (ppm, CDCl₃): 6.96 (dd, J=8.4, 1.9 Hz, 1H), 6.86 (d, J=2.0 Hz, 1H), 6.43 (d, J=8.4 Hz, 1H), 4.91 (s, 1H), 4.10 (t, J=5.8 Hz, 2H), 3.22-3.05 (m, 2H), 2.82 (t, J=5.8 Hz, 2H), 2.68-2.34 (m, 10H), 1.71-1.52 (m, 8H), 1.51-1.39 (m, 4H). ¹³C NMR (75 MHz), δ (ppm, CDCl₃): 146.9, 138.0, 124.0, 114.1, 110.8, 107.3, 66.7, 58.0, 57.3, 54.9, 54.4, 40.3, 26.1, 26.0, 24.5, 24.2. LCMS m/z calc for [M+H]⁺: 410.4, 412.4 found: 410.2, 412.2.

### 4-Bromo-N-[2-(piperidin-1-yl)ethyl]-2-[3-(piperidin-1-yl)propoxy]aniline (9)

Compound 9 was obtained as a brown oil (450 mg, 32%). ¹H NMR (300 MHz), δ (ppm, CDCl₃): 6.95 (dd, J=8.4, 2.1 Hz, 1H), 6.85 (d, J=2.1 Hz, 1H), 6.43 (d, J=8.4 Hz, 1H), 4.81 (t, J=5.0 Hz, 1H), 4.01 (t, J=6.2 Hz, 2H), 3.14 (m, 2H), 2.69-2.25 (m, 12H), 2.11-1.91 (m, 2H), 1.73-1.31 (m, 12H). ¹³C NMR (75 MHz), δ (ppm, CDCl₃): 147.0, 137.8, 123.7, 113.7, 110.8, 107.5, 67.1, 57.5, 57.2, 56.1, 54.7, 40.3, 26.8, 26.1, 25.9, 24.4. LCMS m/z calc for [M+H]⁺: 424.2, 426.2 found: 424.3, 426.2.

### 4-Bromo-2-[2-(piperidin-1-yl)ethoxy]-N-[3-(piperidin-1-yl)propyl]aniline (11)

Compound 11 was obtained as a brown oil (2.30 g, 79%). ¹H NMR (300 MHz), δ (ppm, CDCl₃): 6.95 (dd, J=8.4 Hz, J=2.2 Hz, 1H), 6.86 (d, J=2.2 Hz, 1H), 6.45 (d, J=8.4 Hz, 1H), 4.73 (br s, 1H), 4.09 (t, J=6.2 Hz, 2H), 3.13 (t, J=6.7 Hz, 2H), 2.76 (t, J=6.1 Hz, 2H), 2.51-2.36 (m, 10H), 1.88-1.79 (m, 2H), 1.64-1.57 (m, 8H), 1.49-1.41 (m, 4H). ¹³C NMR (75 MHz), δ (ppm, CDCl₃): 146.2, 138.1, 124.1, 114.4, 110.7, 107.2, 66.6, 57.9, 57.4, 54.8, 42.5, 26.3, 26.0, 24.3. LCMS m/z calc for [M+H]⁺: 424.2, 426.2, found: 424.3, 426.2

### 4-Bromo-2-(3-(piperidin-1-yl)propoxy)-N-(3-(piperidin-1-yl)propyl)aniline (12)

Compound 12 was obtained as a brown oil (940 mg, 56%). ¹H NMR (300 MHz), δ (ppm, CDCl₃): 6.94 (dd, J=8.4, 2.1 Hz, 1H), 6.85 (d, J=2.1 Hz, 1H), 6.44 (d, J=8.4 Hz, 1H), 4.55 (s, 1H), 4.01 (t, J=6.4 Hz, 2H), 3.22-3.03 (m, 2H), 2.55-2.20 (m, 12H), 2.07-1.95 (m, 2H), 1.90-1.76 (m, 2H), 1.70-1.55 (m, 8H), 1.50-1.40 (m, 4H). ¹³C NMR (75 MHz), δ (ppm, CDCl₃): 146.7, 137.7, 123.7, 113.7, 110.6, 107.3, 67.1, 57.4, 55.9, 54.7, 54.6, 42.4, 29.7, 26.8, 26.3, 25.9, 24.4. LCMS m/z calc for [M+H]⁺: 438.2, 440.2, found: 438.3, 440.2.

### 4-Bromo-N-[4-(piperidin-1-yl)butyl]-2-[2-(piperidin-1-yl)ethoxy]aniline (13)

Compound 13 was obtained as a brown oil (513 mg, 55%). ¹H NMR (300 MHz), δ (ppm, CDCl₃): 6.96 (dd, J=8.4, 2.1 Hz, 1H), 6.87 (d, J=2.1 Hz, 1H), 6.44 (d, J=8.4 Hz, 1H), 4.37 (s, 1H), 4.08 (t, J=5.9 Hz, 2H), 3.12 (s, 2H), 2.76 (t, J=5.9 Hz, 2H), 2.58-2.27 (m, 10H), 1.78-1.36 (m, 16H). ¹³C NMR (75 MHz), δ (ppm, CDCl₃): 146.5, 138, 124.2, 114.6, 110.7, 107.1, 66.8, 59.1, 57.9, 55, 54.6, 43.5, 27.4, 26.0, 24.5, 24.2. LCMS m/z calc for [M+H]⁺: 438.2, 440.2, found 438.3, 440.3.

### 4-Bromo-2-(2-cyclohexylethoxy)-N-(3-(piperidin-1-yl)propyl)aniline (14)

Compound 14 was obtained as a brown oil (80 mg, 59%). ¹H NMR (300 MHz), δ (ppm, CDCl₃): 6.96 (dd, J=8.4 Hz, J=2.1 Hz, 1H), 6.83 (d, J=2.4 Hz, 1H), 6.45 (d, J=8.4 Hz, 1H), 3.99 (t, J=6.75 Hz, 2H), 3.13 (t, J=6.6 Hz, 2H), 2.47-2.43 (m, 6H), 1.90-1.60 (m, 13H), 1.50-1.44 (m, 3H), 1.25-1.22 (m, 3H), 1.04-0.97 (m, 2H). ¹³C NMR (75 MHz), δ (ppm, CDCl₃): 146.9, 137.9, 123.5, 113.5, 110.5, 107.4, 66.6, 57.3, 54.7, 42.5, 36.5, 34.8, 33.3, 26.5, 26.2, 26.1, 25.7, 24.3. LCMS m/z calc for [M+H]⁺: 423.2, 425.2, found: 423.2, 425.0.

### 4-Bromo-2-(4-(piperidin-1-yl)butoxy)-N-(2-(piperidin-1-yl)ethyl)aniline (10)

To a solution of 5-bromo-2-([2-(piperidin-1-yl)ethyl]amino)phenol (5) (1 g, 3.34 mmol) in DMF (42 mL), at 0° C., NaH (334 mg, 8.36 mmol) was added. After stirring the mixture for 30 min, 1-bromo-4-chlorobutane (0.43 mL, 3.34 mmol) in DMF (5 mL) was added dropwise at 0° C. The reaction mixture was stirred for another 12 h at rt. The mixture was diluted with 40 mL of water and extracted with DCM (3×60 mL). The combined organic layers were dried over MgSO₄ and evaporated. The residue was purified by column chromatography (DCM/MeOH(NH₃), 9.8:0.2 (v:v)) to afford 4-bromo-2-(4-chlorobutoxy)-N-(2-(piperidin-1-yl)ethyl)aniline as a white oil (450 mg). ¹H NMR (300 MHz), δ (ppm, CDCl₃): 6.97 (dd, J=8.3, 2.1 Hz, 1H), 6.84 (s, 1H), 6.44 (dd, J=8.3, 1.5 Hz, 1H), 4.88 (s, 1H), 4.06-3.97 (m, 2H), 3.70-3.58 (m, 2H), 3.21-3.07 (m, 2H), 2.61 (t, J=5.2 Hz, 2H), 2.48-2.35 (m, 4H), 2.12-1.94 (m, 4H), 1.68-1.44 (m, 6H). ¹³C NMR (75 MHz), δ (ppm, CDCl₃): 146.8, 137.9, 123.9, 113.6, 110.9, 107.4, 67.4, 57.1, 54.3, 44.7, 40.2, 29.4, 26.7, 26.2, 24.5. LCMS m/z calc for [M+H]⁺: 389.1, 391.1, 393.1, found: 389.1, 391.1, 393.2.

4-Bromo-2-(4-chlorobutoxy)-N-(2-(piperidin-1-yl)ethyl)aniline (438 mg, 1.12 mmol) was dissolved in piperidine (11 mL) and was refluxed for 12 h. The mixture was concentrated in vacuo and the residue was dissolved in 1N NaOH (20 mL) and extracted with DCM (3×60 mL). The combined organic layers were dried over MgSO₄, filtrated and concentrated in vacuo. The residue was purified by column chromatography (DCM/MeOH(NH₃), 9.8:0.2 (v:v)) to afford 10 as a brown oil (246 mg, 18% over two steps). ¹H NMR (300 MHz), δ (ppm, CDCl₃): 6.95 (dd, J=8.4, 2.1 Hz, 1H), 6.83 (d, J=2.1 Hz, 1H), 6.43 (d, J=8.4 Hz, 1H), 4.81 (s, 1H), 3.98 (t, J=6.3 Hz, 2H), 3.27-2.98 (m, 2H), 2.60 (t, J=6.3 Hz, 2H), 2.49-2.27 (m, 10H), 2.00-1.78 (m, 2H), 1.76-1.52 (m, 10H), 1.50-1.39 (m, 4H). ¹³C NMR (75 MHz), δ (ppm, CDCl₃): 147.0, 137.9, 123.6, 113.6, 110.7, 107.5, 68.3, 59.0, 57.2, 54.6, 54.4, 40.8, 27.5, 26.2, 25, 24.5, 23.5. LCMS m/z calc for [M+H]⁺: 438.2, 439.2, found: 438.3, 440.3.

### General Procedure for the Synthesis of Compounds 15-23 is Exemplified by the Protocol Used for the Synthesis of 15

2-Formylbenzeneboronic acid (63 mg, 0.42 mmol) was dissolved in a mixture of toluene (8 mL) and EtOH (3.2 mL). K₂CO₃ (62 mg, 0.45 mmol) and 4-bromo-2-[2-(piperidin-1-yl)ethoxy]-N-[3-(piperidin-1-yl)propyl]aniline (11) (150 mg, 0.35 mmol) were added and the reaction was stirred for 30 min and deoxygenated by passing a stream of N₂ through it. Pd₂dba₃ (7 mg, 0.01 mmol) and P(o-tol)₃ (22 mg, 0.07 mmol) were added and the mixture was refluxed for 18 h. After cooling to rt, the mixture was poured into water, extracted with ethyl acetate. The combined organic layers were dried over MgSO₄ and evaporated.

The residue was purified by flash chromatography (PE/EtOAc/MeOH(NH₃), 10:0:0 to 5.2:4.4:0.4 (v/v)) to afford 3′-(2-(piperidin-1-yl)ethoxy)-4′-((3-(piperidin-1-yl)propyl)amino)-[1,1′-biphenyl]-2-carbaldehyde (15) as a brown oil (146 mg, 92%). ¹H NMR (300 MHz), δ (ppm, CDCl₃): 10.03 (s, 1H), 7.99-7.96 (m, 1H); 7.62-7.58 (m, 1H); 7.47-7.38 (m, 2H), 6.87 (dd, J=7.9 Hz, J=2.0 Hz, 1H,), 6.82 (d, J=1.9 Hz, 1H), 6.67 (d, J=8.0 Hz, 1H), 4.98 (br s, 1H), 4.16 (t, J=6.2 Hz, 2H), 3.25 (t, J=6.7 Hz, 2H), 2.81 (t, J=6.1 Hz, 2H), 2.53-2.41 (m, 10H), 1.94-1.85 (m, 2H), 1.68-1.58 (m, 8H), 1.49-1.43 (m, 4H). ¹³C NMR (75 MHz), δ (ppm, CDCl₃): 193.2, 146.6, 145.8, 139.0, 133.8, 133.3, 130.6, 127.5, 126.7, 125.0, 124.2, 113.0, 109.3, 66.6, 58.0, 57.4, 54.9, 42.4, 26.4, 25.9, 24.3. LCMS m/z calc for [M+H]⁺: 450.3, found: 450.1.

### 3′-(2-(piperidin-1-yl)ethoxy)-4′-((3-(piperidin-1-yl)propyl)amino)-[1,1′-biphenyl]-3-carbaldehyde (16)

Compound 16 was obtained as a brown oil (600 mg, 96%). ¹H NMR (300 MHz), δ (ppm, CDCl₃): 10.07 (s, 1H), 8.05 (t, J=1.6 Hz, 1H), 7.81 (m, 1H), 7.75 (dt, J=7.7 Hz, J=1.3 Hz, 1H), 7.55 (t, J=7.7 Hz, 1H), 7.18 (dd, J=8.2 Hz, J=2.0 Hz, 1H), 7.07 (d, J=2.0 Hz, 1H), 6.68 (d, J=8.2 Hz, 1H), 4.95 (br s, 1H), 4.22 (t, J=6.2 Hz, 2H), 3.24 (t, J=6.7 Hz, 2H), 2.83 (t, J=6.1 Hz, 2H), 2.56-2.41 (m, 10H), 1.91-1.87 (m, 2H); 1.67-1.59 (m, 8H), 1.49-1.45 (m, 4H). ¹³C NMR (75 MHz), δ (ppm, CDCl₃): 192.7, 146.4, 142.5, 139.1, 136.8, 132.2, 129.3, 127.3, 120.4, 110.0, 66.6, 58.1, 57.4, 55.1, 54.7, 42.4, 26.4, 26.0, 24.3. LCMS m/z calc for [M+H]⁺: 450.3, found: 450.3.

### 3′-(2-(piperidin-1-yl)ethoxy)-4′-((3-(piperidin-1-yl)propyl)amino)-[1,1′-biphenyl]-4-carbaldehyde (17)

Compound 17 was obtained as a brown oil (285 mg, 88%). ¹H NMR (300 MHz), δ (ppm, CDCl₃): 10.01 (s, 1H), 7.89 (d, J=8.1 Hz, 2H), 7.69 (d, J=8.4 Hz, 2H), 7.23 (dd, J=8.4 Hz, J=2.1, 1H), 7.10 (d, J=2.1 Hz, 1H), 6.67 (d, J=8.4 Hz, 1H), 5.05 (br s, 1H), 4.23 (t, J=6.3 Hz, 2H), 3.27 (t, J=6.6 Hz, 2H), 2.86 (t, J=6.0 Hz, 2H), 2.61-2.39 (m, 10H), 1.93 (m, 2H), 1.75-1.58 (m, 8H), 1.53-1.43 (m, 4H). ¹³C NMR (75 MHz), δ (ppm, CDCl₃): 191.9, 147.6, 146.2, 139.6, 134.0, 133.3, 127.0, 126.4, 121.1, 110.0, 66.5, 57.9, 57.2, 54.8, 42.1, 25.8, 25.5, 24.1. LCMS m/z calc for [M+H]⁺: 450.3, found: 450.2.

### 3′-(2-(piperidin-1-yl)ethoxy)-4′-((2-(piperidin-1-yl)ethyl)amino)-[1,1′-biphenyl]-2-carbaldehyde (18)

Compound 18 was obtained as a brown oil (459 mg, 92%). ¹H NMR (300 MHz), δ (ppm, CDCl₃): 10.03 (s, 1H), 7.98 (dd, J=7.8, 1.4 Hz, 1H), 7.59 (tt, J=13.1, 6.5 Hz, 1H), 7.45-7.41 (m, 2H), 6.87 (dd, J=8.0, 1.9 Hz, 1H), 6.81 (d, J=1.9 Hz, 1H), 6.66 (d, J=8.0 Hz, 1H), 5.13 (s, 1H), 4.16 (t, J=5.8 Hz, 2H), 3.25 (m, 2H), 2.85 (t, J=5.7 Hz, 2H), 2.66 (t, J=6.3 Hz, 2H), 2.59-2.50 (m, 4H), 2.49-2.40 (m, 4H), 1.67-1.39 (m, 12H). ¹³C NMR (75 MHz), δ (ppm, CDCl₃): 193.2, 146.7, 146.1, 139, 133.8, 133.3, 130.6, 127.5, 126.7, 125.2, 124.1, 109.4, 66.6, 58.1, 57.4, 55, 54.4, 40.2, 26.1, 26, 24.5, 24.2. LCMS m/z calc for [M+H]⁺: 436.3, found 436.3.

### 4′-(2-(piperidin-1-yl)ethyl)amino)-3′-(3-(piperidin-1-yl)propoxy)-[1,1′-biphenyl]-2-carbaldehyde (19)

Compound 19 was obtained as a brown oil (378 mg, 92%). ¹H NMR (300 MHz), δ (ppm, CDCl₃): 10.04 (s, 1H), 7.98 (dd, J=7.8, 1.3 Hz, 1H), 7.65-7.54 (m, 1H), 7.50-7.37 (m, 2H), 6.86 (dd, J=8.0, 1.8 Hz, 1H), 6.80 (d, J=1.8 Hz, 1H), 6.66 (d, J=8.0 Hz, 1H), 5.02 (t, J=4.9 Hz, 1H), 4.07 (t, J=6.2 Hz, 2H), 3.25 (dd, J=11.5, 6.0 Hz, 2H), 2.66 (t, J=6.3 Hz, 2H), 2.57-2.32 (m, 10H), 1.67-1.54 (m, 8H), 1.52-1.41 (m, 4H). ¹³C NMR (75 MHz), δ (ppm, CDCl₃): 193.3, 146.7, 146.2, 138.8, 133.8, 133.3, 130.7, 127.5, 126.7, 125.2, 123.8, 112.2, 109.3, 66, 57.3, 56.2, 54.7, 54.4, 40.2, 26, 26.2, 25.9, 24.5, 24.4. LCMS m/z calc for [M+H]⁺: 450.6, found 450.38

### 3′-(4-(piperidin-1-yl)butoxy)-4′-((2-(piperidin-1-yl)ethyl)amino)-[1,1′-biphenyl]-2-carbaldehyde (20)

Compound 20 was obtained as a brown oil (150 mg, 71%). ¹H NMR (300 MHz), δ (ppm, CDCl₃): 10.04 (s, 1H), 7.98 (dd, J=7.8, 1.3 Hz, 1H), 7.65-7.56 (m, 1H), 7.49-7.38 (m, 2H), 6.86 (dd, J=8.0, 1.8 Hz, 1H), 6.78 (d, J=1.8 Hz, 1H), 6.66 (d, J=8.0 Hz, 1H), 5.01 (d, J=4.8 Hz, 1H), 4.04 (t, J=6.2 Hz, 2H), 3.31-3.18 (m, J=11.6, 4.6 Hz, 2H), 2.66 (t, J=6.3 Hz, 2H), 2.54-2.24 (m, 10H), 1.94-1.79 (m, 4H), 1.79-1.53 (m, 8H), 1.53-1.37 (m, 4H). ¹³C NMR (75 MHz), δ (ppm, CDCl₃): 193.3, 146.8, 146.2, 138.8, 133.8, 133.3, 130.7, 127.5, 126.7, 125.2, 123.7, 112.2, 109.4, 68.2, 59.1, 57.3, 54.6, 54.4, 40.3, 26.2, 25.9, 24.5, 23.5. LCMS m/z calc for [M+H]⁺: 464.3, found 464.4.

### 3′-(3-(piperidin-1-yl)propoxy)-4′-((3-(piperidin-1-yl)propyl)amino)-[1,1′-biphenyl]-2-carbaldehyde (21)

Compound 21 was obtained as a brown oil (573 mg, 80%). ¹H NMR (300 MHz), δ (ppm, CDCl₃): 10.02 (s, 1H), 7.98 (d, J=7.6 Hz, 1H), 7.59 (t, J=7.5 Hz, 1H), 7.49-7.36 (m, 2H), 6.85 (dd, J=8.0, 1.7 Hz, 1H), 6.80 (s, 1H), 6.67 (d, J=8.0 Hz, 1H), 4.79 (s, 1H), 4.07 (t, J=6.4 Hz, 2H), 3.25 (d, J=6.2 Hz, 2H), 2.58-2.33 (m, 12H), 2.07-1.99 (m, 2H), 1.96-1.79 (m, 2H), 1.61 (m, 8H), 1.51-1.41 (m, 4H). ¹³C NMR (75 MHz), δ (ppm, CDCl₃): 193.3, 146.8, 145.9, 138.7, 133.8, 133.3, 130.7, 127.5, 125.1, 123.8, 112.3, 109.2, 67.0, 57.4, 56.0, 54.8, 54.7, 42.4, 26.9, 26.5, 25.9, 24.5. LCMS m/z calc for [M+H]⁺:464.3, found 464.4.

### 4′-((4-(piperidin-1-yl)butyl)amino)-3′-(2-(piperidin-1-yl)ethoxy)-[1,1′-biphenyl]-2-carbaldehyde (22)

Compound 22 was obtained as a brown oil (439 mg, 97%). ¹H NMR (300 MHz), δ (ppm, CDCl₃): 10.02 (s, 1H), 7.98 (dd, J=7.8, 1.5 Hz, 1H), 7.59 (td, J=7.5, 1.5 Hz, 1H), 7.45 (dd, J=8.0, 1.2 Hz, 1H), 7.40 (d, J=7.5 Hz, 1H), 6.86 (dd, J=8.0, 1.9 Hz, 1H), 6.82 (d, J=1.9 Hz, 1H), 6.66 (d, J=8.0 Hz, 1H), 4.61 (s, 1H), 4.14 (t, J=5.9 Hz, 2H), 3.21 (d, J=6.1 Hz, 2H), 2.80 (t, J=6.0 Hz, 2H), 2.63-2.26 (m, 10H), 1.77-1.38 (m, 16H). ¹³C NMR (75 MHz), δ (ppm, CDCl₃): 193.2, 146.6, 145.7, 139.0, 133.8, 133.3, 130.6, 127.5, 126.7, 125.0, 124.2, 113.1, 109.2, 66.6, 59.1, 58.0, 55.0, 54.6, 43.4, 27.5, 26.0, 24.6, 24.5, 24.2. LCMS m/z calc for [M+H]⁺: 464.65, found 464.45

### 3′-(2-cyclohexylethoxy)-4′-((3-(piperidin-1-yl)propyl)amino)-[1,1′-biphenyl]-3-carbaldehyde (23)

Compound 23 was obtained as a brown oil (748 mg, 80%). ¹H NMR (300 MHz), δ (ppm, CDCl₃): 10.03 (s, 1H), 7.99 (d, J=7.4 Hz, 1H), 7.60-7.57 (m, 1H), 7.48-7.41 (m, 2H), 6.86-6.66 (m, 3H), 4.77 (br s, 1H), 4.06 (t, J=6.8 Hz, 2H), 3.26 (m, 2H), 2.48 (m, 6H), 2.46-0.97 (m, 21H). ¹³C NMR (75 MHz), δ (ppm, CDCl₃): 193.3, 146.8, 146.0, 138.6, 133.8, 133.3, 130.6, 127.5, 126.6, 125.2, 123.6, 112.0, 109.1, 66.5, 57.4, 54.7, 42.4, 36.7, 34.8, 33.3, 26.4, 26.2, 25.7, 24.3. LCMS m/z calc for [M+H]⁺: 449.3, found 449.4.

### General Procedure for the Synthesis of Compounds 24-30 is Exemplified by the Protocol Used for the Synthesis of 24

### Example 1: 2-[2-(Piperidin-1-yl)ethoxy]-N-[3-(piperidin-1-yl)propyl]-4-[4-(piperidin-1-ylmethyl)phenyl]aniline (Compound 24)

3′-(2-(piperidin-1-yl)ethoxy)-4′-((3-(piperidin-1-yl)propyl)amino)-[1,1′-biphenyl]-4-carbaldehyde 17 (290 mg, 0.65 mmol) was dissolved in DCE (12 mL). Piperidine (96 μL, 0.97 mmol) was added. After 2 h at rt, NaBH(OAc)₃ (206 mg, 0.97 mmol) and acetic acid (56 μL, 0.97 mmol) were added. After 16 h at rt, saturated NaHCO₃ solution was added. The reaction mixture was stirred for another hour. DCM was added and the layers were separated. The organic layer was washed three times with saturated NaHCO₃ solution. The organic layer was dried over MgSO₄ and evaporated. The residue was purified by flash chromatography (DCM/MeOH(NH₃), 10:0 to 9:1 (v/v)) to afford 24 as a colorless oil (260 mg, 77%). ¹H NMR (300 MHz), δ (ppm, CDCl₃): 7.51 (d, J=8.5 Hz, 2H), 7.32 (d, J=8.5 Hz, 2H), 7.13 (dd, J=8.1 Hz, J=1.9 Hz, 1H), 7.04 (d, J=1.8 Hz, 1H), 6.66 (d, J=8.2 Hz, 1H), 4.20 (t, J=6.1 Hz, 2H), 3.50 (s, 2H), 3.22 (t, J=6.7 Hz, 2H), 2.81 (t, J=6.0 Hz, 2H), 2.54-2.34 (m, 14H), 1.93-1.84 (m, 2H), 1.66-1.38 (m, 18H). ¹³C NMR (75 MHz), δ (ppm, CDCl₃): 146.2, 140.2, 138.3, 136.1, 129.6, 129.1, 126.0, 120.2, 110.1, 66.6, 63.6, 58.1, 57.5, 55.1, 54.5, 42.5, 26.6, 26.0, 24.4. LCMS m/z calc for [M+H]⁺: 519.4, found: 519.3.

### Example 2: 4-(4-[(Dimethylamino)methyl]phenyl)-2-[2-(piperidin-1-yl)ethoxy]-N-[3-(piperidin-1-yl)propyl]aniline (Compound 25)

Compound 25 was synthesized according to the general procedure for the synthesis of compounds 24-30 starting from 17 and dimethylamine 2M in THF. Compound 25 was obtained as a colorless oil (130 mg, 61%). ¹H NMR (300 MHz), δ (ppm, CDCl₃): 7.49 (d, J=8.4 Hz, 2H), 7.31 (d, J=8.4 Hz, 2H), 7.12 (dd, J=8.2 Hz, J=1.9 Hz, 1H), 7.04 (d, J=2.1 Hz, 1H), 6.65 (d, J=8.1 Hz, 1H), 4.20 (t, J=6.1 Hz, 2H), 3.45 (s, 2H), 3.23 (t, J=6.8 Hz, 2H), 2.82 (t, J=6.1 Hz, 2H), 2.55-2.41 (m, 10H), 2.25 (s, 3H), 1.90-1.85 (m, 2H), 1.66-1.58 (m, 8H), 1.51-1.44 (m, 4H). ¹³C NMR (75 MHz), δ (ppm, CDCl₃): 146.2, 140.4, 138.3, 136.4, 129.5, 129.0, 126.4, 120.2, 110.0, 66.4, 64.1, 58.1, 57.4, 54.8, 45.3, 42.5, 26.4, 25.8, 24.3. LCMS m/z calc for [M+H]⁺: 479.3, found: 479.4.

### Example 3: 2-[2-(1-piperidyl)ethoxy]-4-[3-(1-piperidylmethyl)phenyl]-N-[3-(1-piperidyl)propyl]aniline (Compound 26)

Compound 26 was synthesized according to the general procedure for the synthesis of compounds 24-30 starting from 16 and piperidine. Compound 26 was obtained as ab oil (17 mg, 5%). ¹H NMR (300 MHz), δ (ppm, CDCl₃): 7.49-7.47 (m, 1H), 7.45-7.43 (m, 1H), 7.33 (t, J=7.5 Hz, 1H), 7.24-7.20 (m, 1H), 7.5 (dd, J=8.1 Hz, J=1.8 Hz, 1H), 7.06 (d, J=1.8 Hz, 1H), 6.66 (d, J=8.1 Hz, 1H), 4.22 (t, J=6.0 Hz, 2H), 3.54 (s, 2H), 3.23 (t, J=6.9 Hz, 2H), 2.83 (t, J=6.3 Hz, 2H), 2.6-2.4 (m, 14H); 1.89 (quint, J=6.6 Hz, 2H), 1.70-1.50 (m, 12H), 1.50-1.40 (m, 6H). ¹³C NMR (75 MHz), δ (ppm, CDCl₃):

146.2, 141.4, 138.4, 129.2, 128.4, 127.3, 127.0, 125.0, 120.3, 110.3, 110.0, 66.6, 64.0, 58.1, 57.4, 55.1, 54.7, 54.5, 42.5, 26.5, 26.0, 25.9, 24.4, 24.2. LCMS m/z calc for [M+H⁺]: 519.4, Found: 519.3.

### Example 4: 2-[2-(Piperidin-1-yl)ethoxy]-N-[3-(piperidin-1-yl)propyl]-4-[2-(piperidin-1-ylmethyl)phenyl]aniline (compound 27)

Compound 27 was synthesized according to the general procedure for the synthesis of compounds 24-30 starting from 15 and piperidine. Compound 27 was obtained as a colorless oil (114 mg, 66%). ¹H NMR (300 MHz), δ (ppm, CDCl₃): 7.54-7.51 (m, 1H), 7.29-7.25 (m, 3H), 6.93 (d, J=1.8 Hz, 1H), 6.89 (dd, J=8.0 Hz, J=1.8 Hz, 1H), 6.64 (d, J=8.0 Hz, 1H), 4.98 (br s, 1H), 4.15 (t, J=6.1 Hz, 2H), 3.42 (s, 2H), 3.23 (t, J=6.8 Hz, 2H), 2.80 (t, J=6.1 Hz, 2H), 2.52-2.30 (m, 14H), 1.90 (quint, J=6.7 Hz, 2H), 1.68-1.38 (m, 18H). ¹³C NMR (75 MHz), δ (ppm, CDCl₃): 145.3, 143.1, 137.1, 136.1, 130.1, 129.5, 126.3, 122.8, 113.0, 109.3, 66.4, 60.8, 58.1, 57.4, 54.9, 54.4, 42.6, 26.6, 26.0, 24.3. LCMS m/z calc for [M+H]⁺: 519.4, found: 519.2.

### Example 5: 4-[2-(Morpholin-4-ylmethyl)phenyl]-2-[2-(piperidin-1-yl)ethoxy]-N-[3-(piperidin-1-yl)propyl]aniline (Compound 28)

Compound 28 was synthesized according to the general procedure for the synthesis of compounds 24-30 starting from 15 and morpholine. Compound 28 was obtained as a colorless oil (126 mg, 40%). ¹H NMR (300 MHz), δ (ppm, CDCl₃): 7.53-7.48 (m, 1H); 7.29-7.26 (m, 3H), 6.94-6.89 (m, 2H), 6.64 (d, J=8.6 Hz, 1H), 4.80 (br s, 1H), 4.14 (t, J=6.1 Hz, 2H), 3.68 (t, J=4.4 Hz, 4H); 3.45 (s, 2H); 3.23 (t, J=6.8 Hz, 2H); 2.79 (t, J=6.0 Hz, 2H); 2.52-2.38 (m, 14H), 1.90 (quint, J=6.7 Hz, 2H), 1.66-1.56 (m, 8H), 1.50-1.42 (m, 4H). ¹³C NMR (75 MHz), δ (ppm, CDCl₃): 145.4, 143.3, 137.8, 135.1, 130.3, 129.2, 126.5, 122.8, 112.9, 109.3, 67.2, 66.3, 60.5, 58.1, 57.5, 54.9, 53.4, 42.6, 26.6, 26.0, 24.3. LCMS m/z calc for [M+H]⁺: 521.4, Found: 521.4.

### Example 6: 4-(2-[(4-Methylpiperazin-1-yl)methyl]phenyl)-2-[2-(piperidin-1-yl)ethoxy]-N-[3-(piperidin-1-yl)propyl]aniline (Compound 29)

Compound 29 was synthesized according to the general procedure for the synthesis of compounds 24-30 starting from 15 and N-methylpiperazine. Compound 29 was obtained as a colorless oil (213 mg, 54%). ¹H NMR (300 MHz), δ (ppm, CDCl₃): 7.48-7.45 (m, 1H), 7.27-7.24 (m, 3H), 6.94-6.90 (m, 2H), 6.63 (d, J=8.0 Hz, 1H), 4.13 (t, J=6.1 Hz, 2H), 3.44 (s, 2H), 3.22 (t, J=6.8 Hz, 2H), 2.79 (t, J=6.1 Hz, 2H), 2.50-2.27 (m, 21H), 1.88 (quint, J=7.5 Hz, 2H), 1.66-1.56 (m, 8H), 1.48-1.42 (m, 4H). ¹³C NMR (75 MHz), δ (ppm, CDCl₃): 145.3, 143.3, 137.8, 135.5, 130.3, 129.3, 126.4, 122.8, 113.0, 109.2, 66.5, 60.1, 58.1, 57.5, 55.0, 46.1, 42.6, 26.7, 26.0, 24.3). LCMS m/z calc for [M+H^{+]:} 534.2, found: 534.4.

### Example 7: 4-(2-[(Dimethylamino)methyl]phenyl)-2-[2-(piperidin-1-yl)ethoxy]-N-[3-(piperidin-1-yl)propyl]aniline (Compound 30)

Compound 30 was synthesized according to the general procedure for the synthesis of compounds 24-30 starting from 15 and 13 equiv. of dimethylamine 2M in THF. Compound 30 was obtained as a colorless oil (162 mg, 51%). ¹H NMR (300 MHz), δ (ppm, CDCl₃): 7.51-7.49 (m, 1H), 7.30-7.25 (m, 3H), 6.91 (d, J=1.8 Hz, 1H), 6.85 (dd, J=8.0 Hz, J=1.8 Hz, 1H), 6.65 (d, J=8.0 Hz, 1H), 4.74 (br s, 1H), 4.14 (t, J=6.2 Hz, 2H), 3.39 (s, 2H), 3.23 (t, J=6.7 Hz, 2H), 2.80 (t, J=6.1 Hz, 2H), 2.54-2.40 (m, 10H), 2.18 (s, 6H), 1.93-1.84 (m, 2H), 1.67-1.57 (m, 8H), 1.50-1.42 (m, 4H). ¹³C NMR (75 MHz), δ (ppm, CDCl₃): 145.4, 142.9, 137.7, 136.3, 130.0, 129.4, 126.5, 122.7, 112.9, 109.3, 66.3, 61.1, 58.1, 57.5, 54.9, 45.4, 42.6, 26.6, 26.0, 24.3. LCMS m/z calc for [M+H⁺]: 479.3, Found: 479.3.

### General Procedure for the Synthesis of Compounds 31-36 is Exemplified by the Protocol Used for the Synthesis of 31

### Example 8: 3-(2-cyclohexylethoxy)-2′-((dimethylamino)methyl)-N-(3-(piperidin-1-yl)propyl)-[1,1′-biphenyl]-4-amine (Compound 31)

To a solution of 3′-(2-cyclohexylethoxy)-4′-((3-(piperidin-1-yl)propyl)amino)-[1,1′-biphenyl]-3-carbaldehyde 23 (300 mg, 0.67 mmol) and dimethylamine hydrochloride (110 mg, 1.34 mmol) in EtOH (2 mL) was added triethylamine (186 μL, 1.34 mmol) and titanium isopropoxide (400 μL, 1.34 mmol). The mixture was stirred for 12 h at rt. NaBH₄ (51 mg, 1.34 mmol) was then added and the mixture was further stirred for 2 hours. The reaction was quenched by pouring the mixture into aqueous ammonia (2.5 mL) and extracted with DCM (3×10 mL). The combined organic layers were dried over MgSO₄ and evaporated. The residue was purified by flash chromatography (DCM/MeOH(NH₃), 10:0 to 9:1 (v/v)) to afford 31 as a colorless oil (157 mg, 49%). ¹H NMR (300 MHz), δ (ppm, CDCl₃): 7.53-7.49 (m, 1H), 7.33-7.28 (m, 3H), 6.91 (d, J=1.8 Hz, 1H), 6.85 (dd, J=8.0 Hz, J=1.8 Hz, 1H), 6.65 (d, J=8.0 Hz, 1H), 4.64 (br s, 1H), 4.05 (t, J=6.8 Hz, 2H), 3.40 (s, 2H), 3.24 (t, J=6.4 Hz, 2H), 2.49-2.40 (m, 6H), 2.18 (s, 6H), 1.94-1.85 (m, 2H), 1.80-1.60 (m, 11H), 1.54-1.42 (m, 3H), 1.33-1.10 (m, 3H), 1.06-0.92 (m, 2H). ¹³C NMR (75 MHz), δ (ppm, CDCl₃): 145.6, 143.0, 137.4, 136.3, 130.2, 129.9, 129.5, 126.6, 126.4, 122.2, 112.2, 109.1, 66.3, 61.1, 57.6, 54.8, 45.4, 42.7, 36.8, 34.8, 33.4, 26.6, 26.5, 26.2, 25.9, 24.4. LCMS m/z calc for [M+H]⁺: 478.4, Found: 478.3.

### Example 9: 2′-((Dimethylamino)methyl)-3-(3-(piperidin-1-yl)propoxy)-N-(3-(piperidin-1-yl)propyl)-[1,1′-biphenyl]-4-amine (Compound 32)

Compound 32 was synthesized according to the general procedure for the synthesis of compounds 31-36 starting from compound 21. Compound 32 was obtained as a brown oil (75 mg, 47%). ¹H NMR (300 MHz), δ (ppm, CDCl₃): 7.54-7.47 (m, 1H), 7.35-7.24 (m, 3H), 6.91 (d, J=1.8 Hz, 1H), 6.85 (dd, J=8.0, 1.8 Hz, 1H), 6.64 (d, J=8.1 Hz, 1H), 4.58 (s, 1H), 4.05 (t, J=6.5 Hz, 2H), 3.39 (s, 2H), 3.23 (t, J=6.7 Hz, 2H), 2.59-2.34 (m, 12H), 2.19 (s, 6H), 2.09-1.97 (m, 2H), 1.95-1.82 (m, 2H), 1.69-1.53 (m, 8H), 1.51-1.39 (m, 4H). ¹³C NMR (75 MHz), δ (ppm, CDCl₃): 145.5, 143.0, 137.4, 136.4, 130.2, 129.9, 129.6, 126.6, 126.4, 122.4, 112.4, 109.2, 66.9, 61.1, 57.6, 56.2, 54.8, 54.7, 45.4, 42.6, 27.0, 26.7, 26.0, 24.5. LCMS m/z calc for [M+H]⁺: 493.4, found: 493.4.

### Example 10: 2′-((Dimethylamino)methyl)-3-(2-(piperidin-1-yl)ethoxy)-N-(2-(piperidin-1-yl)ethyl)-[1,1′-biphenyl]-4-amine (Compound 33)

Compound 33 was synthesized according to the general procedure for the synthesis of compounds 31-36 starting from compound 18. Compound 33 was obtained as a brown oil (25 mg, 30%). ¹H NMR (300 MHz), δ (ppm, CDCl₃): 7.51 (dd, J=4.7, 2.3 Hz, 1H), 7.33-7.24 (m, 3H), 6.91 (d, J=1.8 Hz, 1H), 6.87 (dd, J=7.9, 1.8 Hz, 1H), 6.63 (d, J=8.0 Hz, 1H), 4.96 (s, 1H), 4.15 (t, J=5.8 Hz, 2H), 3.40 (s, 2H), 3.25 (t, J=6.1 Hz, 2H), 2.84 (t, J=5.8 Hz, 2H), 2.73-2.39 (m, 10H), 2.19 (s, 6H), 1.66-1.56 (m, 8H), 1.46 (d, J=5.1 Hz, 4H). ¹³C NMR (75 MHz), δ (ppm, CDCl₃): ¹³C NMR (75 MHz, CDCl₃) δ 145.59, 142.95, 137.63, 136.40, 130.17, 129.91, 129.49, 126.59, 126.47, 122.65, 112.71, 109.33, 77.45, 77.03, 76.60, 66.41, 61.11, 58.21, 57.65, 54.93, 54.46, 45.39, 40.37, 26.14, 26.03, 24.50, 24.21. LCMS m/z calc for [M+H]⁺: 465.3, found 465.4.

### Example 11: 2′-((Dimethylamino)methyl)-N-(2-(piperidin-1-yl)ethyl)-3-(3-(piperidin-1-yl)propoxy)-[1,1′-biphenyl]-4-amine (Compound 34)

Compound 34 was synthesized according to the general procedure for the synthesis of compounds 31-36 starting from compound 19. Compound 34 was obtained as a brown oil (36 mg, 30%). ¹H NMR (300 MHz), δ (ppm, CDCl₃): 7.58-7.43 (m, 1H), 7.37-7.24 (m, 3H), 6.92 (d, J=1.8 Hz, 1H), 6.86 (dd, J=8.0, 1.8 Hz, 1H), 6.63 (d, J=8.0 Hz, 1H), 4.86 (s, 1H), 4.05 (t, J=6.2 Hz, 2H), 3.40 (s, 2H), 3.33-3.16 (m, 2H), 2.66 (t, J=6.3 Hz, 2H), 2.59-2.32 (m, 10H), 2.19 (s, 6H), 2.10-1.97 (m, 2H), 1.70-1.53 (m, 8H), 1.53-1.40 (m, 4H). ¹³C NMR (75 MHz), δ (ppm, CDCl₃): 145.7, 143.0, 137.5, 136.4, 130.2, 129.9, 129.6, 126.6, 126.5, 122.4, 112.3, 109.3, 66.8, 61.2, 57.6, 56.3, 54.7, 54.5, 45.4, 40.4, 27.1, 26.2, 26.0, 24.5. LCMS m/z calc for [M+H]⁺: 479.4, found: 479.4.

### Example 12: 2′-((Dimethylamino)methyl)-3-(4-(piperidin-1-yl)butoxy)-N-(2-(piperidin-1-yl)ethyl)-[1,1′-biphenyl]-4-amine (Compound 35)

Compound 35 was synthesized according to the general procedure for the synthesis of compounds 31-36 starting from compound 20. Compound 35 was obtained as a brown oil (35 mg, 28%). ¹H NMR (300 MHz), δ (ppm, CDCl₃): 7.61-7.41 (m, 1H), 7.40-7.19 (m, 3H), 6.90 (d, J=1.6 Hz, 1H), 6.86 (dd, J=8.0, 1.6 Hz, 1H), 6.63 (d, J=8.0 Hz, 1H), 4.86 (s, 1H), 4.16-3.91 (m, 2H), 3.36 (d, J=18.2 Hz, 2H), 3.32-3.16 (m, 2H), 2.66 (t, J=6.3 Hz, 2H), 2.39 (dd, J=19.5, 12.2 Hz, 10H), 2.19 (s, 6H), 1.96-1.33 (m, 16H). ¹³C NMR (75 MHz), δ (ppm, CDCl₃): 145.7, 143.0, 137.5, 136.4, 130.2, 129.9, 129.5, 126.6, 126.4, 122.3, 112.3, 109.3, 68.0, 61.2, 59.2, 57.6, 54.6, 54.5, 45.4, 40.5, 27.7, 26.2, 26.0, 24.5, 24.5, 23.6. LCMS m/z calc for [M+H]⁺: 493.4, found 493.5.

### Example 13: 2′-((Dimethylamino)methyl)-N-(4-(piperidin-1-yl)butyl)-3-(2-(piperidin-1-yl)ethoxy)-[1,1′-biphenyl]-4-amine (Compound 36)

Compound 36 was synthesized according to the general procedure for the synthesis of compounds 31-36 starting from compound 22. Compound 36 was obtained as a brown oil (75 mg, 47%). ¹H NMR (300 MHz), δ (ppm, CDCl₃): 7.54-7.47 (m, 1H), 7.35-7.24 (m, 3H), 6.91 (d, J=1.8 Hz, 1H), 6.85 (dd, J=8.0, 1.8 Hz, 1H), 6.64 (d, J=8.1 Hz, 1H), 4.58 (s, 1H), 4.05 (t, J=6.5 Hz, 2H), 3.39 (s, 2H), 3.23 (t, J=6.7 Hz, 2H), 2.59-2.34 (m, 12H), 2.19 (s, 6H), 2.09-1.97 (m, 2H), 1.95-1.82 (m, 2H), 1.69-1.53 (m, 8H), 1.51-1.39 (m, 4H). ¹³C NMR (75 MHz), δ (ppm, CDCl₃): 145.5, 143.0, 137.4, 136.4, 130.2, 129.9, 129.6, 126.6, 126.4, 122.4, 112.4, 109.2, 66.9, 61.1, 57.6, 56.2, 54.8, 54.7, 45.4, 42.6, 27.0, 26.7, 26.0, 24.5. LCMS m/z calc for [M+H]⁺: 493.4, found: 493.4.

### Example 14: 3-(2-(Piperidin-1-yl)ethoxy)-N-(3-(piperidin-1-yl)propyl)-[1,1′-biphenyl]-4-amine (Compound 37)

To a solution of benzeneboronic acid (108 mg, 0.882 mmol) in a mixture of toluene (8 mL) and EtOH (3 mL), K₂CO₃ (132 mg, 0.956 mmol) and 11 (156 mg, 0.368 mmol) were added. The reaction was stirred for 30 min and deoxygenated by passing a stream of N₂ through it. Pd₂dba₃ (6.73 mg, 0.007 mmol) and P(o-tol)₃ (22 mg, 0.073 mmol) were added and the mixture was refluxed overnight. After cooling, the mixture was poured into 1N NaOH and extracted three times with dichloromethane. The combined organic layers were dried over MgSO₄ and evaporated. The residue was purified by flash chromatography (DCM/MeOH(NH₃), 10:0 to 95:5 (v/v)) to afford 37 as a brown oil (110 mg, 0.261 mmol, 71%). ¹H NMR (300 MHz), δ (ppm, CD₂Cl₂): 7.61-7.53 (m, 2H), 7.44-7.36 (m, 2H), 7.30-7.22 (m, 1H), 7.15 (dd, J=8.2, 2.0 Hz, 1H), 7.09 (d, J=2.0 Hz, 1H), 6.68 (d, J=8.2 Hz, 1H), 4.94 (s, 1H), 4.18 (t, J=6.0 Hz, 2H), 3.24 (t, J=6.7 Hz, 2H), 2.78 (t, J=6.0 Hz, 2H), 2.60-2.31 (m, 10H), 1.91-1.75 (m, 2H), 1.70-1.55 (m, 8H), 1.55-1.38 (m, 4H). ¹³C NMR (75 MHz), δ (ppm, CD₂Cl₂): 148.3, 143.4, 140.7, 130.5, 128.0, 127.8, 122.0, 112.2, 111.6, 68.6, 60.0, 59.1, 56.9, 56.6, 44.2, 28.2, 28, 27.8, 26.3, 26.2. LCMS m/z calc for [M+H]⁺: 422.3, found: 422.3.

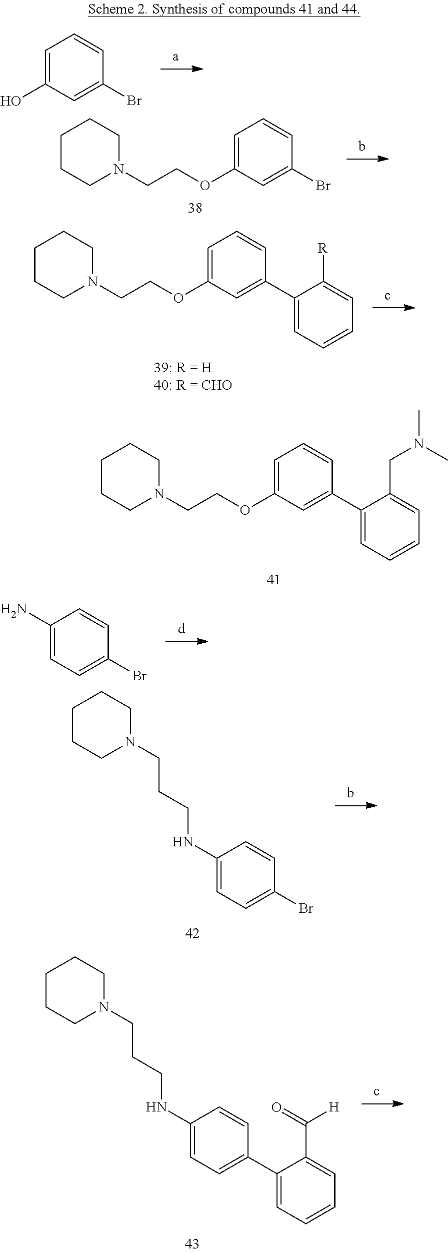

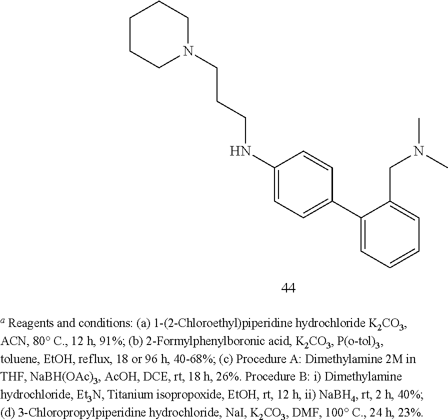 [SMILES: CN(C)Cc1ccccc1-c1ccc(NCCCN2CCCCC2)cc1]

### 1-(2-(3-Bromophenoxy)ethyl)piperidine (38)

3-Bromophenol (1.63 g, 9.42 mmol) was suspended in ACN (50 mL) and K₂CO₃ (3.91 g, 28.3 mmol) was added. The reaction mixture was stirred at 80° C. for 30 min. Then 1-(2-chloroethyl)piperidine hydrochloride (2.43 g, 13.2 mmol) was added. The reaction mixture was stirred at 80° C. for 12 h. The inorganics were removed by filtration and the solvent was evaporated. The residue was dissolved in DCM (50 mL) and washed twice with a 1N NaOH solution. The organic layers were combined and dried over MgSO₄, filtrated and concentrated in vacuo to afford 38 (2.6 g, 91%). The product was used without further purification in the next step. ¹H NMR (300 MHz), δ (ppm, CDCl₃): 7.14 (d, J=8.1 Hz, 1H), 7.08-7.04 (m, 2H), 6.83 (ddd, J=8.1, 2.5, 1.4 Hz, 1H), 4.08 (t, J=6.1 Hz, 2H), 2.75 (t, J=6.1, 2H), 1.65-1.53 (m, 6H), 1.49-1.39 (m, 2H). LCMS m/z calc for [M+H]⁺: 286.1, 288.1 found: 286.0, 288.0

### 3′-(2-(Piperidin-1-yl)ethoxy)-[1,1′-biphenyl]-2-carbaldehyde (40)

To a solution of 2-formylbenzeneboronic acid (893 mg, 5.953 mmol) in a mixture of toluene (113 mL) and EtOH (45 mL), K₂CO₃ (891 mg, 6.449 mmol) and 38 (1.4 g, 4.961 mmol) were added. The reaction was stirred for 30 min and deoxygenated by passing a stream of N₂ through it. Pd₂dba₃ (91 mg, 0.02 mmol) and P(o-tol)₃ (302 mg, 0.992 mmol) were added and the mixture was refluxed for 2 h. After cooling, the mixture was poured into a 1N NaOH solution and extracted three times with dichloromethane. The combined organic layers were dried over MgSO₄ and evaporated to afford 40 as an orange oil which was used without further purification (1.85 g, 83%). ¹H NMR (300 MHz), δ (ppm, CDCl₃): 10.00 (d, J=0.8 Hz, 1H), 8.04 (d, J=7.7 Hz, 1H), 7.63 (d, J=7.5 Hz, 1H), 7.50-7.34 (m, 3H), 6.99-6.93 (m, 3H), 4.17 (t, J=5.9 Hz, 2H), 2.83 (t, J=5.9 Hz, 2H), 2.56 (m, 4H), 1.65-1.60 (m, 4H), 1.50-1.44 (m, 2H). LCMS m/z calc for [M+H]⁺: 310.1, found: 310.12.

### Example 15: N,N-Dimethyl-1-(3′-(2-(piperidin-1-yl)ethoxy)-[1,1′-biphenyl]-2-yl)methanamine (compound 41)

To a solution of 40 (925 mg, 2.99 mmol) in DCE (37 mL), dimethylamine 2.0 M in THF (15 mL, 29.9 mmol), NaBH(OAc)₃ (950 mg, 4.484 mmol) and acetic acid (0.26 mL, 4.484 mmol) were added and the mixture was stirred at rt for 12 h. The reaction was quenched by pouring the mixture into a saturated NaHCO₃ solution (25 mL) and extracted with DCM (3×50 mL). The combined organic layers were dried over MgSO₄ and evaporated. The residue was purified by flash chromatography (DCM/MeOH(NH₃), 10:0 to 97:3 (v/v)) to afford 41 as a brown oil (266 mg, 0.786 mmol, 26%). ¹H NMR (300 MHz, MeOD) 7.51 (dd, J=7.4, 1.6 Hz, 1H), 7.39-7.27 (m, 3H), 7.25-7.19 (m, 1H), 6.95 (m, 1H), 6.92-6.84 (m, 2H), 4.17 (t, J=5.7 Hz, 2H), 3.45 (s, 2H), 2.80 (t, J=5.6 Hz, 2H), 2.57 (d, J=5.2 Hz, 4H), 2.10 (s, 6H), 1.64 (m, 4H), 1.50 (m, 2H). ¹³C NMR (75 MHz), δ (ppm, CDCl₃): 158.4, 142.9, 142.2, 136.3, 129.8, 128.8, 127.3, 126.6, 122.1, 122.1, 115.9, 113.1, 66, 30.9, 58, 55.1, 45.4, 26, 24.2. LCMS m/z calc for [M+H]⁺: 339.2, found: 339.2.

### 4-Bromo-N-(2-(piperidin-1-yl)ethyl)aniline (42)

To solution of 4-bromoaniline (1.34 mL, 12 mmol) in DMF (50 mL), K₂CO₃ (3.2 g, 23 mmol), NaI (8.7 g, 58 mmol) and 3-chloropropylpiperidine hydrochloride (3.4 g, 18 mmol) were added. The reaction mixture was stirred at 100° C. for 24 h. DMF was removed under reduced pressure and the residue was taken up in 20 mL of water. The aqueous layer was extracted with DCM (3×60 mL). The combined organic layers were dried over MgSO₄ and evaporated. The residue was purified by column chromatography (EtOAc/MeOH, 8.5:1.5 (v:v)) to afford 42 as a brown oil (779 mg, 23%). ¹H NMR (300 MHz), δ (ppm, CDCl₃): 7.23 (d, J=8.8 Hz, 2H), 6.47 (d, J=8.8 Hz, 1H), 3.15 (t, J=6.2 Hz, 2H), 2.67-2.41 (m, 6H), 1.97-1.76 (m, 2H), 1.77-1.44 (m, 6H). ¹³C NMR (75 MHz), δ (ppm, CDCl₃): 147.7, 131.9, 114.1, 108.4, 57.7, 54.5, 43.4, 29.7, 25.5, 24. LCMS m/z calc for [M+H]⁺: 283.1, 285.1 found: 283.1, 285.1

### 4′-((3-(piperidin-1-yl)propyl)amino)-[1,1′-biphenyl]-2-carbaldehyde (43)

To a solution of 2-formylbenzeneboronic acid (791 mg, 5.275 mmol) in a mixture of toluene (35 mL) and EtOH (17 mL), K₂CO₃ (724 mg, 5.239 mmol) and 42 (779 mg, 2.621 mmol) were added. The reaction was stirred for 30 min and deoxygenated by passing a stream of N₂ through it. Pd₂dba₃ (133 mg, 0.055 mmol) and P(o-tol)₃ (395 mg, 0.485 mmol) were added and the mixture was refluxed for 96 h. After cooling, the mixture was poured into water and extracted with dichloromethane. The combined organic layers were dried over MgSO₄ and evaporated. The residue was purified by semi-preparative chromatography (eluent H₂O formic, ACN formic, 100:0 to 70:30 (v/v)) to afford 43 as an orange oil (341 mg, 40%). ¹H NMR (300 MHz), δ (ppm, CDCl₃): 10.02 (d, J=0.7 Hz, 1H), 8.57 (s, 1H), 7.98 (dd, J=7.7, 1.0 Hz, 1H), 7.64-7.55 (m, 1H), 7.47-7.35 (m, 2H), 7.22-7.14 (m, 2H), 6.71 (d, J=8.6 Hz, 2H), 3.34-3.23 (m, 2H), 3.11-2.99 (m, 6H), 2.13 (dd, J=13.9, 6.5 Hz, 2H), 1.97-1.84 (m, 4H), 1.72-1.53 (m, 2H). ¹³C NMR (75 MHz), δ (ppm, CDCl₃): 193.2, 168.3, 148.2, 146.4, 133.6, 133.4, 131.3, 130.6, 127.5, 126.7, 126, 112.3, 55.1, 53.1, 40.9, 23.8, 23.1, 22.6. LCMS m/z calc for [M+H]⁺: 323.2, found 323.2.

### Example 16: 2′-((Dimethylamino)methyl)-N-(3-(piperidin-1-yl)propyl)-[1,1′-biphenyl]-4-amine (Compound 44)

To a solution of 43 (156 mg, 0.423 mmol) and dimethylamine hydrochloride (69 mg, 0.847 mmol) in EtOH (1 mL) was added triethylamine (0.12 mL, 0.847 mmol) and titanium isopropoxide (0.25 mL, 0.847 mmol). The mixture was stirred at rt for 12 h. NaBH₄ (32 mg, 0.847 mmol) was added and the resulting mixture was further stirred for 2 h. The reaction was quenched by pouring the mixture into aqueous ammonia (2.5 mL) and extracted with DCM (3×10 mL). The combined organic layers were dried over MgSO₄ and evaporated. The residue was purified by flash chromatography (DCM/MeOH(NH₃), 10:0 to 98:2 (v/v)) to afford 44 as a brown oil (59 mg, 40%). ¹H NMR (300 MHz), δ (ppm, CDCl₃): 7.60-7.45 (m, 1H), 7.37-7.23 (m, 3H), 7.20 (d, J=8.6 Hz, 2H), 6.65 (d, J=8.6 Hz, 2H), 5.08 (s, 1H), 3.41 (s, 2H), 3.24 (t, J=6.3 Hz, 2H), 2.47 (dd, J=14.0, 7.5 Hz, 6H), 2.18 (s, 6H), 1.96-1.74 (m, 2H), 1.74-1.57 (m, 4H), 1.49 (d, J=5.1 Hz, 2H). ¹³C NMR (75 MHz), δ (ppm, CDCl₃): 147.8, 142.7, 136.5, 130.5, 130.1, 130, 129.7, 126.6, 126.5, 112.0, 61.0, 58.3, 54.7, 45.4, 43.9, 26.2, 25.8, 24.5. LCMS m/z calc for [M+H]⁺: 352.3, found 352.3.

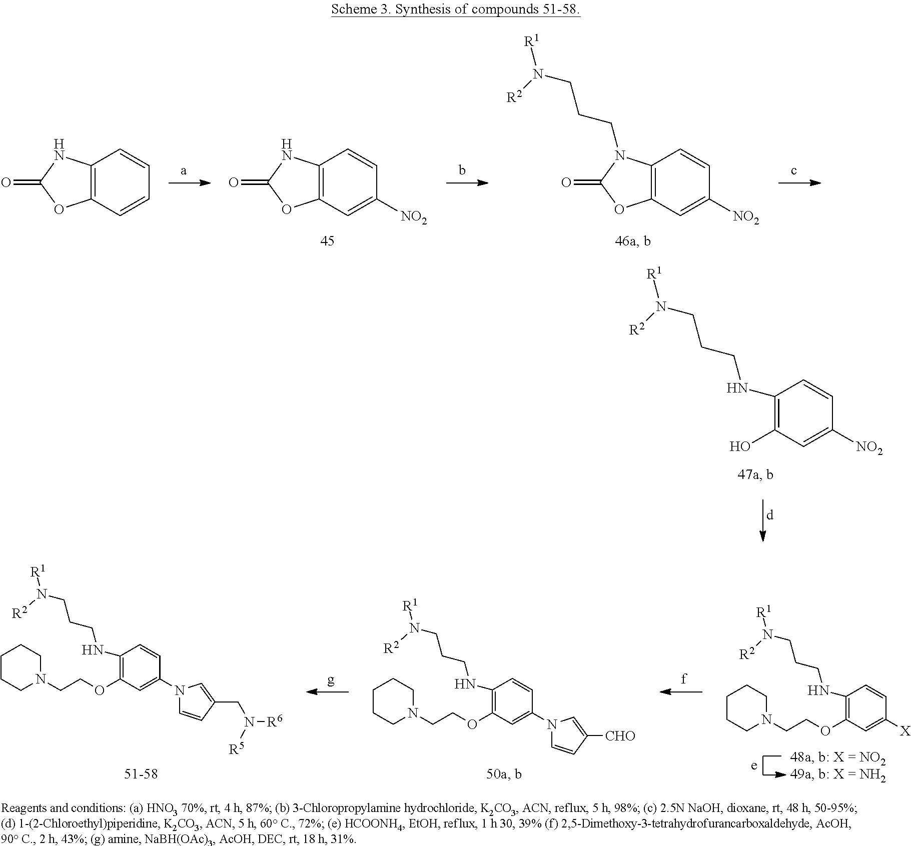

### 6-Nitro-3H-1,3-benzoxazol-2-one (45)

Nitric acid (50 mL, 0.80 mol) was cooled to 0° C. and 3H-1,3-benzoxazol-2-one (5.00 g, 37 mmol) was added. The reaction mixture was stirred at rt for 4 h and then poured in ice. The resulting precipitate was collected by filtration, washed with water and dried to give 45 as a pink solid (5.80 g, 87%). Mp 250° C. ¹H NMR (300 MHz), δ (ppm, DMSO-d₆): 12.41 (s, 1H), 8.20 (d, J=2.2 Hz, 1H), 8.13 (dd, J=8.6 Hz, J=2.4 Hz, 1H), 7.28 (d, J=8.6 Hz, 1H). ¹³C NMR (75 MHz), δ (ppm, DMSO-d₆): 154.7, 143.2, 142.5, 137.2, 121.2, 109.8, 105.8. LCMS m/z calc for [M−H]⁺: 179.0, found: 179.0.

### 6-Nitro-3-[3-(piperidin-1-yl)propyl]-1,3-benzoxazol-2-one (46a)

6-Nitro-3H-1,3-benzoxazol-2-one (45) (6.30 g, 35 mmol) was suspended in ACN (150 mL) and K₂CO₃ (6.91 g, 50 mmol) was added. The reaction mixture was stirred at 80° C. for 30 min. 3-Chloropropylpiperidine hydrochloride (3.96 g, 20 mmol) was added and the reaction mixture was stirred at 80° C. for another 5 h. The inorganics were removed by filtration and the solvent was evaporated. The residue was purified by flash chromatography (DCM/MeOH(NH₃), 9.8:0.2 (v/v)) to give compound 46a as a beige solid (5 g, 98%). Mp 81° C. ¹H NMR (300 MHz), δ (ppm, CDCl₃): 8.21 (dd, J=8.7 Hz, J=2.1 Hz, 1H), 8.10 (d, J=2.1 Hz, 1H), 7.25 (d, J=8.5 Hz, 1H), 4.00 (t, J=6.5 Hz, 2H), 2.38-2.28 (m, 6H), 2.02-1.98 (m, 2H), 1.56-1.53 (m, 4H), 1.45-1.41 (m, 2H). ¹³C NMR (75 MHz), δ (ppm, CDCl₃): 154.1, 143.0, 141.9, 137.3, 120.7, 108.0, 106.1, 55.4, 54.4, 41.1, 25.9, 24.4, 24.3. LCMS m/z calc for [M+H]⁺: 306.2, found: 306.2.

### 3-[3-(Dimethylamino)propyl]-6-nitro-1,3-benzoxazol-2-one (46b)

Same procedure as for the preparation of compound 46a was followed but with 3-chloropropyldimethylamine. Yield: 77% (7.10 g, yellow solid). Mp: 44.8° C. ¹H NMR (300 MHz), δ (ppm, CDCl₃): 8.20 (dd, J=8.7 Hz, J=2.2 Hz, 1H); 8.07 (d, J=2.2 Hz, 1H); 7.26 (d, J=8.7 Hz, 1H), 3.96 (t, J=6.7 Hz, 2H); 2.31 (t, J=6.9 Hz, 2H); 2.18 (s, 6H); 1.94 (m, 2H). ¹³C NMR (75 MHz), δ (ppm, CDCl₃): 153.1; 142.1; 140.9; 136.1; 119.9; 106.9; 105.2; 54.9; 44.2; 39.8; 24.5. LCMS m/z calc for [M+H⁺]: 266.1, found: 266.0.

### 5-Nitro-2-([3-(piperidin-1-yl)propyl]amino)phenol (47a)

To a solution of 6-nitro-3-[3-(piperidin-1-yl)propyl]-1,3-benzoxazol-2-one (46a) (1 g, 3.28 mmol) in dioxane (5 mL), aqueous 2.5N NaOH (26 mL, 65.5 mmol) was added. The mixture was stirred at 20° C. for 48 h. After cooling to 0° C., a 1N HCl solution was added to reach pH 8. After 30 min of stirring, the mixture was extracted with EtOAc (3×100 mL). The combined organic layers were dried over MgSO₄ and evaporated. The residue was purified by flash chromatography (DCM/MeOH(NH₃), 9.5:0.5 (v/v)) to give compound 47a as an orange solid (715 mg, 78%). Mp: 152° C. ¹H NMR (300 MHz), δ (ppm, CDCl₃): 7.69 (dd, J=8.7 Hz, J=2.4 Hz, 1H), 7.49 (d, J=2.4 Hz, 1H), 6.32 (d, J=8.8 Hz, 1H), 3.23 (t, J=6.5 Hz, 2H); 2.67-2.57 (m, 6H), 1.95-1.91 (m, 2H), 1.71-1.67 (m, 4H), 1.53-1.48 (m, 2H). ¹³C NMR (75 MHz), δ (ppm, CDCl₃): 145.4, 144.3, 136.7, 118.5, 108.8, 106.4, 56.3, 54.1, 41.2, 25.1, 24.5, 23.5. LCMS m/z calc for [M+H]⁺: 280.2, found: 280.1.

### 2-([3-(Dimethylamino)propyl]amino)-5-nitrophenol (47b)

Same procedure as for the preparation of compound 47a was followed but starting from 46b. Yield: 60% (540 mg, red solid). Mp: 147.6° C. ¹H NMR (300 MHz), δ (ppm, CDCl₃): 7.73 (dd, J=8.8 Hz, J=2.4 Hz, 1H); 7.42 (d, J=2.4 Hz, 1H); 6.38 (d, J=8.8 Hz, 1H); 3.24 (t, J=7.4 Hz, 2H); 2.44 (t, J=7.4 Hz, 2H); 2.26 (s, 6H); 1.85 (m, 2H). ¹³C NMR (75 MHz), δ (ppm, CDCl₃): 144.5; 143.1; 136.5; 120.0; 108.1; 106.5; 56.3; 44.8; 40.5; 26.3. LCMS m/z calc for [M+H⁺]: 240.1, found: 240.0.

### 4-Nitro-2-[2-(piperidin-1-yl)ethoxy]-N-[3-(piperidin-1-yl)propyl]aniline (48a)

5-Nitro-2-([3-(piperidin-1-yl)propyl]amino)phenol (47a) (200 mg, 0.72 mmol) was suspended in ACN (6 mL) and K₂CO₃ (300 mg, 2.15 mmol) was added. The reaction mixture was stirred at rt for 10 min. 1-(2-Chloroethyl)piperidine hydrochloride (160 mg, 0.86 mmol) was then added and the reaction mixture was stirred at 60° C. for another 5 h. The inorganics were removed by filtration and the solvent was evaporated. The residue was purified by flash chromatography (DCM/MeOH(NH₃), 9.8:0.2 (v/v)) to give 48a as a brown oil (200 mg, yield 72%). ¹H NMR (300 MHz), δ (ppm, CDCl₃): 7.86 (dd, J=8.9 Hz, J=2.4 Hz, 1H), 7.62 (d, J=2.4 Hz, 1H), 6.46 (d, J=9.2 Hz, 1H), 6.19 (br t, J=4.9 Hz, 1H), 4.17 (t, J=6.1 Hz, 2H), 3.28 (m, 2H), 2.77 (t, J=5.8 Hz, 2H), 2.48 (m, 4H), 2.45-2.33 (m, 6H), 1.85-1.82 (m, 2H), 1.65-1.54 (m, 8H), 1.49-1.39 (m, 4H). ¹³C NMR (75 MHz), δ (ppm, CDCl₃): 145.1, 144.3, 136.3, 120.3, 106.48, 106.3, 66.4, 57.6, 57.2, 54.8, 54.5, 42.0, 25.8, 25.7, 24.2. LCMS m/z calc for [M+H]⁺: 391.3, found: 391.1.

### N-[3-(Dimethylamino)propyl]-4-nitro-2-[2-(piperidin-1-yl)ethoxy]aniline (48b)

2-([3-(Dimethylamino)propyl]amino)-5-nitrophenol (47b) (0.50 g, 2.10 mmol), 1-piperidineethanol (0.42 mL, 3.14 mmol) and triphenylphosphine (1.10 g, 4.18 mmol) were placed in round-bottom flask under N₂ atmosphere. Dry THF (6 mL) was added and the reaction mixture was cooled to 0° C. After 10 min of stirring, DEAD (0.66 mL, 4.18 mmol), was added. The reaction mixture was stirred at 50° C. under nitrogen for 6 h. The solvent was evaporated. The residue was purified by flash chromatography (DCM/MeOH(NH₄OH 2.5%), 9.8:0.2 to 9:1 (v/v)) to afford 48b as a yellow oil (520 mg, 71%). ¹H NMR (300 MHz), δ (ppm, CDCl₃): 7.88 (dd, J=8.8 Hz, J=2.2 Hz, 1H); 7.58 (d, J=2.2 Hz, 1H); 6.48 (br m, 1H); 6.43 (d, J=8.8 Hz, 1H); 4.17 (t, J=6.0 Hz, 2H); 3.32 (m, 2H); 2.82 (t, J=6.0 Hz, 2H); 2.52 (m, 4H); 2.45 (t, J=6.5 Hz, 2H); 2.26 (s, 6H); 1.87 (m, 2H); 1.65 (m, 4H); 1.48 (m, 2H). ¹³C NMR (75 MHz), δ (ppm, CDCl₃): 145.2; 144.3; 136.5; 120.3; 106.3; 66.1; 57.8; 54.7; 45.5; 42.3; 26.1; 25.7; 24.0. LCMS m/z calc for [M+H⁺]: 351.2, found: 351.0.

### 2-[2-(Piperidin-1-yl)ethoxy]-N-[3-(piperidin-1-yl)propyl]benzene-1,4-diamine (49a)

4-Nitro-2-[2-(piperidin-1-yl)ethoxy]-N-[3-(piperidin-1-yl)propyl]aniline (48a) (100 mg, 0.255 mmol) was dissolved in MeOH (5 mL). Pd/C 10% (20 mg, 7 mol %) was added and the reaction mixture was stirred at 40° C. for 30 min. The catalyst was filtered through a celite pad and the filtrate was evaporated to give 49a as a purple oil which was used in the next step without further purification. ¹H NMR (300 MHz), δ (ppm, CDCl₃): 6.43 (d, J=8.2 Hz, 1H), 6.30-6.20 (m, 2H), 4.03 (t, J=6.3 Hz, 2H), 3.63 (br s, 3H), 3.06 (t, J=7.1 Hz), 2.74 (t, J=6.1 Hz, 2H), 2.47-2.44 (m, 4H), 2.42-2.31 (m, 6H), 1.78-1.73 (m, 2H), 1.62-1.52 (m, 8H), 1.47-1.35 (m, 4H). ¹³C NMR (75 MHz), δ (ppm, CDCl₃): 147.2, 137.3, 131.8, 111.8, 108.0, 111.4, 66.3, 58.0, 57.5, 55.0, 54.7, 43.5, 26.7, 26.0, 24.3. LCMS m/z calc for [M+H]⁺: 361.3, found: 361.1.

### 1-(3-[2-(Piperidin-1-yl)ethoxy]-4-([3-(piperidin-1-yl)propyl]amino)phenyl)-1H-pyrrole-3-carbaldehyde (50a)

2-[2-(Piperidin-1-yl)ethoxy]-N-[3-(piperidin-1-yl)propyl]benzene-1,4-diamine (49a) was dissolved in acetic acid (5 mL). 2,5-Dimethoxy-3-formyl-2,3,4,5-tetrahydrofuran (380 μL, 2.71 mmol) was added and the reaction mixture was heated at 90° C. for 2 h. The solvent was then evaporated and the residue taken up in saturated NaHCO₃ solution (50 mL). The aqueous layer was extracted with EtOAc (3×50 mL). The combined organic layers were dried over MgSO₄ and evaporated. The residue was purified by flash chromatography (DCM/MeOH(NH₃), 9.8:0.2 (v/v)) to afford 50a as a brown oil (410 mg, 43%). ¹H NMR (300 MHz), δ (ppm, CDCl₃): 9.81 (s, 1H), 7.54 (dd, J=2.1 Hz, J=1.7 Hz, 1H), 6.95 (dd, J=2.9 Hz, J=2.2 Hz, 1H), 6.89 (dd, J=8.2 Hz, J=2.3 Hz, 1H), 6.81 (d, J=2.3 Hz, 1H), 6.74 (dd, J=2.8 Hz, J=1.8 Hz, 1H), 6.59 (d, J=8.4 Hz, 1H), 4.91 (br s, 1H), 4.15 (t, J=6.1 Hz, 2H), 3.21 (t, J=6.6 Hz, 2H), 2.80 (t, J=6.2 Hz, 2H), 2.59-2.35 (m, 10H), 1.84-1.89 (m, 2H), 1.69-1.53 (m, 8H), 1.53-1.38 (m, 4H). ¹³C NMR (75 MHz), δ (ppm, CDCl₃): 185.5, 146.3, 138.5, 129.0, 127.7, 127.3, 123.0, 114.8, 109.3, 108.9, 105.8, 66.8, 57.9, 57.4, 55.0, 54.7, 42.5, 26.2, 25.9, 24.2. LCMS m/z calc for [M+]⁺: 439.3, found: 439.2.

### 1-(4-([3-(Dimethylamino)propyl]amino)-3-[2-(piperidin-1-yl)ethoxy]phenyl)-1H-pyrrole-3-carbaldehyde (50b)

N-[3-(Dimethylamino)propyl]-4-nitro-2-[2-(piperidin-1-yl)ethoxy]aniline (48b) (112 mg, 0.32 mmol) was dissolved in dry EtOH (10 mL). A catalytic amount of Pd/C 10% and ammonium formate (210 mg, 3.36 mmol) were added and the reaction mixture was stirred at 80° C. under N₂ for 1 h 30. The catalyst was quickly eliminated by filtration through a celite pad and the filtrate was evaporated. The purple oil was dissolved in acetic acid (1 mL). Then 2,5-dimethoxy-3-formyl-2,3,4,5-tetrahydrofuran (56 μL, 0.4 mmol) was added and the reaction mixture was heated at 90° C. for 2 h. The solvent was evaporated. The residue was purified by flash chromatography (DCM/MeOH(NH₄OH 2.5%), 9.9:0.1 to 9:1 (v/v)) to afford 50b as a yellow oil (50 mg, 39%). ¹H NMR (300 MHz), δ (ppm, CDCl₃): 9.80 (s, 1H); 7.54 (dd, J=2.0 Hz, J=1.7 Hz, 1H); 6.96 (dd, J=3.0 Hz, J=2.0 Hz, 1H); 6.90 (dd, J=8.3 Hz, J=2.3 Hz, 1H); 6.80 (d, J=2.3 Hz, 1H); 6.73 (dd, J=3.0 Hz, J=1.7 Hz, 1H); 6.60 (d, J=8.3 Hz, 1H); 5.00 (br s, 1H); 4.16 (t, J=5.8, 2H); 3.24 (t, J=6.9 Hz, 2H); 2.83 (t, J=5.8 Hz, 2H); 2.53 (m, 4H); 2.46 (t, J=6.9 Hz, 2H); 2.27 (s, 6H); 1.88 (m, 2H); 1.65 (m, 4H); 1.49 (m, 2H). ¹³C NMR (75 MHz), δ (ppm, CDCl₃): 185.6; 146.2; 138.5; 129.0; 127.8; 127.3; 123.1; 114.9; 109.2; 105.6; 66.2; 57.8; 54.9; 45.4; 42.2; 26.9; 25.8; 24.1. LCMS m/z calc for [M+H⁺]: 399.3, found: 399.0.

### Example 17: 4-(3-[(Dimethylamino)methyl]-1H-pyrrol-1-yl)-2-[2-(piperidin-1-yl)ethoxy]-N-[3-(piperidin-1-yl) propyl]aniline (51)

To a solution of 1-(3-[2-(piperidin-1-yl)ethoxy]-4-([3-(piperidin-1-yl)propyl]amino)phenyl)-1H-pyrrole-3-carbaldehyde (50a) (85 mg, 0.19 mmol) in DCE (2 mL), dimethylamine in THF (2M, 144 μL, 0.29 mmol) was added. After 1 h 30 of stirring at rt, NaBH(OAc)₃ (61 mg, 0.29 mmol) and acetic acid (17 μL, 0.29 mmol) were added. After 5 h at rt, saturated NaHCO₃ solution was added. The reaction mixture was stirred for another 1 h. DCM was added and the layers were separated. The organic layer was washed twice with saturated NaHCO₃ solution. The organic layer was dried over MgSO₄ and evaporated. The residue was purified by semi-preparative chromatography (eluent H₂O formic, ACN formic, 9:1 to 1:9 (v/v)) to afford compound 51 as a yellow oil (28 mg, 31%). ¹H NMR (300 MHz), δ (ppm, CDCl₃): 6.91-6.85 (m, 3H), 6.82 (d, J=2.3 Hz, 1H), 6.58 (d, J=8.4 Hz, 1H), 6.23 (dd, J=2.0 Hz, J=2.1 Hz, 1H), 4.68 (br s, 1H), 4.15 (t, J=6.2 Hz, 2H), 3.40 (s, 2H), 3.19 (t, J=6.7 Hz, 2H), 2.80 (t, J=6.2 Hz, 2H), 2.53-2.41 (m, 10H), 2.29 (s, 6H), 1.87-1.84 (m, 2H), 1.65-1.58 (m, 8H), 1.49-1.42 (m, 4H). ¹³C NMR (75 MHz), δ (ppm, CDCl₃): 146.3, 137.1, 130.9, 121.4, 119.5, 113.9, 110.5, 109.7, 105.5, 66.7, 57.9, 57.5, 56.5, 54.9; 45.0, 42.7, 26.5, 26.0, 24.4. LCMS m/z calc for [M+H]⁺: 468.4, found: 468.3.

### General Procedure for the Synthesis of Compounds 52-58

1-(3-[2-(Piperidin-1-yl)ethoxy]-4-([3-(piperidin-1-yl or dimethylamino)propyl]amino)phenyl)-1H-pyrrole-3-carbaldehyde (50a-b) was dissolved in DCE (2 mL). The corresponding substituted amine (0.27 mmol) was added. After 1 h 30 of stirring at 20° C., NaBH(OAc)₃ (58 mg, 0.27 mmol) and acetic acid (16 μL, 0.27 mmol) were added. After 16 h of stirring at 20° C., saturated NaHCO₃ solution was added. The reaction mixture was stirred for 1 h. DCM was added and the layers were separated. The organic layer was washed twice with saturated NaHCO₃ solution. The organic layer was dried over MgSO₄ and evaporated. The residue was purified by flash chromatography (DCM/MeOH(NH₃), 10:0 to 9:1 (v/v)) to afford compounds 52-58.

### Example 18: 2-[2-(Piperidin-1-yl)ethoxy]-N-[3-(piperidin-1-yl)propyl]-4-[3-(piperidin-1-ylmethyl)-1H-pyrrol-1-yl]aniline (Compound 52)

Compound 52 was synthesized according to the general procedure for the synthesis of compounds 52-58 by using piperidine as amine. Compound 52 was obtained as a brown oil (20 mg, 40%). ¹H NMR (300 MHz), δ (ppm, CDCl₃): 6.95 (m, 1H); 6.91 (m, 1H); 6.87 (dd, J=8.3 Hz, J=2.7 Hz, 1H); 6.81 (d, J=2.5 Hz, 1H); 6.58 (d, J=8.6 Hz, 1H); 6.25 (m, 1H); 4.71 (br s, 1H); 4.15 (t, J=6.2 Hz, 2H); 3.58 (s, 2H); 3.20 (t, J=6.3 Hz, 2H); 2.81 (t, J=5.9 Hz, 2H); 2.65-2.36 (M, 14H); 1.95 (m, 2H); 1.77-1.56 (M, 12H); 1.52-1.40 (M, 6H). ¹³C NMR (75 MHz), δ (ppm, CDCl₃): 146.2; 137.5; 130.3; 121.5; 120.5; 116.6; 114.4; 111.6; 109.6; 105.7; 66.4; 57.7; 57.2; 54.7; 52.6; 42.4; 25.8; 24.04. LCMS m/z calc for [M+H⁺]: 508.4, found: 508.2. HR-MS: m/z calculated: 508.40099, found: 508.40132 [M+H]⁺=C₃₁H₅ON₅O. HPLC (C₄, 35 min): t_{R} 10.4 min, P_{HPLC} 97%; HPLC (C₁₈, 35 min): t_{R} 18.5 min, P_{HPLC} 95%.

### Example 19: 4-[3-(Morpholin-4-ylmethyl)-1H-pyrrol-1-yl]-2-[2-(piperidin-1-yl)ethoxy]-N-[3-(piperidin-1-yl)propyl]aniline (Compound 53)

Compound 53 was synthesized according to the general procedure for the synthesis of compounds 52-58 by using morpholine as amine. Compound 53 was obtained as a yellow oil (90 mg, 78%). ¹H NMR (300 MHz), δ (ppm, CDCl₃): 6.92-6.85 (M, 3H); 6.82 (d, J=2.3 Hz, 1H); 6.60 (d, J=8.4 Hz, 1H); 6.24 (m, 1H); 4.70 (br s, 1H); 4.16 (t, J=6.2 Hz, 2H); 3.75 (t, J=4.6 Hz, 4H); 3.46 (s, 2H); 3.20 (t, J=6.7 Hz, 2H); 2.81 (t, J=6.2 Hz, 2H); 2.54-2.39 (M, 14H); 1.86 (m, 2H); 1.66-1.59 (M, 8H); 1.50-1.43 (M, 4H). ¹³C NMR (75 MHz), δ (ppm, CDCl₃): 146.3; 137.2; 130.8; 120.4; 119.5; 114.0; 110.7; 109.7; 105.5; 67.1; 66.8; 57.9; 57.5; 55.9; 55.0; 53.5; 42.7; 26.5; 26.0; 24.3. LCMS m/z calc for [M+H⁺]: 510.4, found: 510.1. HR-MS: m/z calculated: 510.38025, found: 510.38051 [M+H]⁺=C₃₀H₄₈N₅O₂. HPLC (C₄, 35 min): t_{R} 15.4 min, P_{HPLC} 98%; HPLC (C₁₈, 35 min): t_{R} 18.6 min, P_{HPLC} 99%.

### Example 20: 4-(3-[(4-Methylpiperazin-1-yl)methyl]-1H-pyrrol-1-yl)-2-[2-(piperidin-1-yl)ethoxy]-N-[3(piperidin-1-yl)propyl]aniline (Compound 54)

Compound 54 was synthesized according to the general procedure for the synthesis of compounds 52-58 by using N-methylpiperazine as amine. Compound 54 was obtained as a yellow oil (55 mg, 54%). ¹H NMR (300 MHz), δ (ppm, CDCl₃): 6.88-6.83 (M, 3H); 6.80 (d, J=2.3 Hz, 1H); 6.57 (d, J=8.4 Hz, 1H); 6.22 (dd, J=2.2 Hz, J=2.3 Hz, 1H); 4.65 (br s, 1H); 4.14 (t, J=6.2 Hz, 2H); 3.47 (s, 2H); 3.19 (t, J=6.7 Hz, 2H); 2.79 (t, J=6.2 Hz, 2H); 2.53-2.37 (M, 18H); 2.29 (s, 3H); 1.85 (m, 2H); 1.65-1.57 (M, 8H); 1.49-1.41 (M, 4H). ¹³C NMR (75 MHz), δ (ppm, CDCl₃): 146.3; 137.1; 130.9; 120.7; 119.5; 114.0; 110.8; 109.7; 105.5; 66.7; 57.9; 57.5; 55.1; 52.9; 46.0; 42.7; 26.5; 26.0; 24.4. LCMS m/z calc for [M+H⁺]: 523.4, found: 523.3. HR-MS: m/z calculated: 523.41189, found: 523.41254 [M+H]⁺=C₃₁H₅₁N₆O. HPLC (C₄, 35 min): t_{R} 14.5 min, P_{HPLC} 95%; HPLC (C₁₈, 35 min): t_{R} 17.0 min, P_{HPLC} 95%.

### Example 21: 4-(3-[(dimethylamino)methyl]-1H-pyrrol-1-yl)-N-[3-(dimethylamino)propyl]-2-[2-(piperidin-1-yl)ethoxy]aniline (compound 55)

Compound 55 was synthesized according to the general procedure for the synthesis of compounds 52-58 by using dimethylamine as amine. Compound 55 was obtained as a yellow oil (33 mg, 31%). ¹H NMR (300 MHz), δ (ppm, CDCl₃): 6.90-6.89 (M, 2H); 6.88 (dd, J=8.2 Hz, J=2.3 Hz, 1H); 6.81 (d, J=2.3 Hz, 1H); 6.59 (d, J=8.5 Hz, 1H); 6.24 (dd, J=2.3 Hz, J=2.4 Hz, 1H); 4.76 (br s, 1H); 4.15 (t, J=6.2 Hz, 2H); 3.42 (s, 2H); 3.22 (t, J=6.6 Hz, 2H); 2.82 (t, J=6.2 Hz, 2H); 2.51 (m, 4H); 2.43 (t, J=7.0 Hz, 2H); 2.30 (s, 6H); 2.26 (s, 6H); 1.88 (m, 2H); 1.65 (m, 4H); 1.49 (m, 2H). ¹³C NMR (75 MHz), δ (ppm, CDCl₃): 146.3; 137.2; 130.8; 121.1; 119.7; 114.0; 110.6; 109.6; 105.5; 66.6; 58.0; 56.4; 55.0; 45.6; 44.8; 42.5; 27.2; 26.0; 24.2. LCMS m/z calc for [M+H⁺]: 428.3, found: 428.3. HR-MS: m/z Calculated: 428.33839, found: 428.33695 [M+H]⁺=C₂₅H₄₂N₅O. HPLC (C₄, 35 min): t_{R} 14.9 min, P_{HPLC} 92%; HPLC (C₁₈, 35 min): t_{R} 16.8 min, P_{HPLC} 96%.

### Example 22: N-[3-(Dimethylamino)propyl]-2-[2-(piperidin-1-yl)ethoxy]-4-[3-(piperidin-1-ylmethyl)-1H-pyrrol-1-yl]aniline (Compound 56)

Compound 56 was synthesized according to the general procedure for the synthesis of compounds 52-58 by using piperidine as amine. Compound 56 was obtained as a yellow oil (41 mg, 35%). ¹H NMR (300 MHz), δ (ppm, CDCl₃): 6.89-6.88 (M, 2H); 6.87 (dd, J=8.4 Hz, J=2.3 Hz, 1H); 6.81 (d, J=2.3 Hz, 1H); 6.59 (d, J=8.4 Hz, 1H); 6.23 (dd, J=2.2 Hz, J=2.2 Hz, 1H); 4.77 (br s, 1H); 4.15 (t, J=5.9 Hz, 2H); 3.45 (s, 2H); 3.22 (t, J=6.9 Hz, 2H); 2.82 (t, J=5.9 Hz, 2H); 2.53-2.83 (M, 10H); 2.25 (s, 6H); 1.88 (m, 2H); 1.65-1.58 (M, 8H); 1.49-1.43 (M, 4H). ¹³C NMR (75 MHz), δ (ppm, CDCl₃): 146.3; 137.1; 130.1; 120.6; 118.7; 114.0; 111.0; 109.6; 105.5; 67.2; 58.0; 56.1; 55.0; 54.1; 45.6; 42.5; 27.2; 26.0; 24.4. LCMS m/z calc for [M+H⁺]: 468.4, found: 468.1. HR-MS: m/z calc for [M+H⁺]: 468.36969, found: 468.36961=C₂₈H₄₆N₅O. HPLC (C₄, 35 min): t_{R} 15.9 min, P_{HPLC} 94%; HPLC (C₁₈, 35 min): t_{R} 17.7 min, P_{HPLC} 95%.

### Example 23: N-[3-(Dimethylamino)propyl]-4-[3-(morpholine-4-ylmethyl)-1H-pyrrol-1-yl]-2-[2-(piperidin-1-yl)ethoxy]aniline (Compound 57)

Compound 57 was synthesized according to the general procedure for the synthesis of compounds 52-58 by using morpholine as amine. Compound 57 was obtained as a yellow oil (71 mg, 60%). ¹H NMR (300 MHz), δ (ppm, CDCl₃): 6.90-6.88 (M, 2H); 6.86 (dd, J=8.5 Hz, J=2.3 Hz, 1H); 6.80 (d, J=2.3 Hz, 1H); 6.59 (d, J=8.5 Hz, 1H); 6.23 (dd, J=2.3 Hz, J=1.9 Hz, 1H); 4.75 (br s, 1H); 4.15 (t, J=6.2 Hz, 2H); 3.75 (t, J=4.7 Hz, 4H); 3.45 (s, 2H); 3.22 (t, J=6.6 Hz, 2H); 2.82 (t, J=6.2 Hz, 2H); 2.51-2.50 (M, 8H); 2.43 (t, J=7.0 Hz, 2H); 2.26 (s, 6H); 1.87 (m, 2H); 1.65 (m, 4H); 1.49 (m, 2H). ¹³C NMR (75 MHz), δ (ppm, CDCl₃): 146.3; 137.2; 130.8; 120.4; 119.7; 114.0; 110.7; 109.6; 105.5; 67.0; 66.6; 58.0; 55.9; 55.0; 53.5; 45.6; 42.5; 27.1; 26.0; 24.2. LCMS m/z calc for [M+H^{+]:} 470.4, found: 470.3. HR-MS: m/z calculated: 470.34895, found: 470.34762 [M+H]⁺=C₂₇H₄₄N₅O₂. HPLC (C₄, 35 min): t_{R} 14.9 Min, P_{HPLC} 98%; HPLC (C₁₈, 35 min): t_{R} 16.8 min, P_{HPLC} 97%.

### Example 24: N-[3-(dimethylamino)propyl]-4-(3-[(4-methylpiperazin-1-yl)methyl]-1H-pyrrol-1-yl}-2-[2-(piperidin-1-yl)ethoxy]aniline (Compound 58)

Compound 58 was synthesized according to the general procedure for the synthesis of compounds 52-58 by using N-methylpiperazine as amine. Compound 58 was obtained as a yellow oil (61 mg, 50%). ¹H NMR (300 MHz), δ (ppm, CDCl₃): 6.87-6.86 (M, 2H); 6.86 (dd, J=8.5 Hz, J=2.5 Hz, 1H); 6.79 (d, J=2.5 Hz, 1H); 6.58 (d, J=8.5 Hz, 1H); 6.22 (dd, J=2.5 Hz, J=2.1 Hz, 1H); 4.74 (br s, 1H); 4.14 (t, J=6.0 Hz, 2H); 3.46 (s, 2H); 3.21 (t, J=6.7 Hz, 2H); 2.81 (t, J=6.0 Hz, 2H); 2.52-2.49 (M, 12H); 2.42 (t, J=6.7 Hz, 2H); 2.28 (s, 3H); 2.24 (s, 6H); 1.86 (m, 2H); 1.64 (m, 4H); 1.25 (m, 2H). ¹³C NMR (75 MHz), δ (ppm, CDCl₃): 146.3; 137.1; 131.2; 120.7; 119.6; 114.0; 110.8; 109.6; 105.5; 66.7; 58.0; 55.4; 55.1; 52.9; 46.0; 45.6; 42.5; 27.2; 26.0; 24.2. LCMS m/z calc for [M+H⁺]: 483.4, found: 483.2. HR-MS: m/z calculated: 483.38059, found: 483.37962 [M+H]⁺=C₂₈H₄₇N₆O. HPLC (C₄, 35 min): t_{R} 13.5 min, P_{HPLC} 98%; HPLC (C₁₈, 35 min): t_{R} 16.1 min, P_{HPLC} 99%.

### Biological Evaluations

### Materials and Methods

Antibodies. Primary antibodies used in this study for western-blot analysis included a homemade rabbit antiserum against the last 17 amino acids of human APP protein sequence, named APP-Cter-C17 (1/5000 in TBS-M). Anti-β-actin (1/10 000 in TBS-M) was obtained from Sigma. Anti-LC3B (1/2000 in TBS-BSA) and anti-p62/SQSTM1 (1/2000 in TBS-M) antibodies were purchased from Cell Signaling Technology. Secondary antibodies (peroxidase-labeled goat anti-rabbit IgG, 1/5000 or peroxidase-labeled horse anti-mouse IgG, 1/50000 in TBS-T) were obtained from Vector Laboratories.

Cell culture and treatment. The human neuroblastoma cell line SY5Y-APP^{695WT} was maintained in Dulbecco's modified Eagle medium (DMEM, high glucose, pyruvate—GIBCO by Life Technologies) supplemented with 10% foetal bovine serum, 2 mM L-glutamine, 1 mM non-essential amino acids and penicillin/streptomycin (GIBCO by Life Technologies) at 37° C. in a 5% CO₂ humidified incubator. SY5Y-APP^{695WT} cells were maintained in 75 cm² culture flasks (Falcon) with supplemented DMEM medium until they reached 80% of confluence 24 h before treatment.

For compounds treatment, a 10 mM stock solution was diluted in freshly supplemented DMEM medium to obtain the precise final concentration of drug. Bafilomycin A1 (Baf_{A1}), the vacuolar 362 type H+ ATPase inhibitor, was purchased from Merck Millipore and was used at a final dilution of 100 nM.

For drug treatment, cells were plated at a rate of 5·10⁵ cells per well into 12-well plates (Falcon) and cultured with 1 mL supplemented DMEM cell medium for 24 h before drug exposure. The following day, cell medium was replaced with fresh medium containing drugs diluted at the indicated concentrations. Cells were treated for 24 h. At the end of treatments, the cell medium was collected and kept at −80° C. until use for dosage. Then, cells were rinsed once with PBS and extracted in 100 μL of Laemmli buffer (10 mM Tris, 20% glycerol and 2% Sodium dodecyl sulfate) using a cell-scraper. The cell lysate was further sonicated (30 pulses of 0.5 s, 60 Hz) for 5 min. Total protein concentration was determined using the Pierce BCA Protein Assay Kit (Thermo scientific) according to the manufacturer's instructions. Samples were stored at −80° C. until analysis.

Primary neuronal cultures and drug treatment. Primary neuron cultures were obtained from C57BL6 wild-type mice as described previously (Domise, M. et al., Sci. Rep. 2016, 6 1-12). Briefly, females were sacrificed at 18.5 days of gestation to collect forebrains of fetuses. Dissection was performed under a microscope in ice-cold Hank's balanced salt solution containing 0.5% w/v D-glucose and 25 mM Hepes buffer. Cells were dissociated mechanically in dissection medium containing 0.01% w/v papain, 0.1% w/v dispase II, and 0.01% w/v DNase and incubated at 37° C. twice for 10 min. Cells were then centrifuged at 220 g for 5 min at 4° C., re-suspended in Neurobasal medium supplemented with 2% B27, 1 mM sodium pyruvate, 100 units/ml penicillin, 100 μg/ml streptomycin, 2 mM Glutamax (Invitrogen), filtered through a 40 μm cell strainer, counted, and plated on poly-L-ornithine- and laminin-coated 12-well plates at a density of 5·10⁵ cells per well. New medium was added at a ratio of 1:3 of starting volume every 3 days until the end of experiments. Drug treatments with 30 and 31 at 1, 3 or 5 μM were performed directly in the conditioned cell media for 24 h at 16 days in vitro (DIV).

Western Blot Analysis. Cell protein lysates were prepared for western-blot analysis by diluting the sample with 1 volume of NuPAGE® lithium dodecyl sulfate (LDS) 2× sample buffer supplemented with 20% NuPAGE® sample reducing agents (Invitrogen). Samples were heat 10 min at 100° C. 10 μg of total proteins per well were loaded onto a precast 4-12% Criterion XT Bis-Tris polyacrylamide gel (Bio-Rad) and electrophoresis was achieved after applying a tension of 150 V during 90 min using a Criterion electrophoresis Cell with the NuPAGE® MOPS SDS running buffer (1×). Proteins were transferred to a nitrocellulose membrane of 0.4 μM pore size (G&E Healthcare) using the Criterion blotting system and applying a tension of 100 V for 45 min. To resolve proteins of low molecular weights such as carboxy-terminal fragments of APP, 12% Criterion XT Bis-Tris polyacrylamide gels (Bio-Rad) were used and electrophoresis was performed during 70 min at 150 V in a NuPAGE® MES SDS running buffer (1×). Proteins were transferred to a nitrocellulose membrane of 0.2 μm pore size (G&E Healthcare) at 100 V for 40 min. Molecular weights calibration was achieved using molecular weight markers (Novex and Magic Marks, Life Technologies). Protein transfer and quality were determined by a reversible Ponceau Red coloration (0.2% Xylidine Ponceau Red and 3% Trichloroacetic acid). Membranes were then blocked in 25 mM Tris-HCl pH 8.0, 150 mM NaCl, 0.1% Tween-20 (v/v) (TBS-T) and 5% (w/v) of skimmed milk (TBS-M) or 5% (w/v) of bovine serum albumin (TBS-BSA) depending on the antibody during 1 h. Membrane was rinsed three-times 10 min in TBS-T before incubation with the primary antibody overnight at 4° C. Membrane was rinsed 3 times 10 min with TBS-T and then incubated with the secondary antibody for 45 min at room temperature (RT). The immunoreactive complexes were revealed using the ECL™ Western Blotting Detection Reagents (G&E Healthcare) and image acquisitions were performed with the Amersham Imager 600 (G&E Healthcare). Quantifications of protein expression levels were performed with ImageJ Software (NIH).

Quantification of secreted Aβ_{1-40/1-42} and sAPPα/sAPPβ. Conditioned media of SY5Y-APP^{695WT} collected at the end of treatments were centrifuged at 1000 g for 5 min to eliminate cells debris. Aβ₁₋₄₀ and Aβ₁₋₄₂ peptide concentrations in μg/mL were determined using amyloid-beta 40 and 42 Human ELISA kits (Invitrogen) according to the manufacturer's instructions after 1:10 and 1:2 dilutions of supernatants in ELISA buffer, respectively. sAPPα and sAPPβ concentrations in ng/mL were determined using the sAPPα/sAPPβ kit (Meso Scale Diagnostics, MSD®) according to the manufacturer's instructions.

Immunofluorescence. SY5Y-APP^{695WT} cells were plated at 3·10⁵ cells per well on poly-D-lysine coated glass coverslip with 1 mL of DMEM supplemented medium. Primary neurons were plated at 2.5×10⁵ cells par well with 1 mL of Neurobasal supplemented medium. Treatments with 30 at 5 μM for SY5Y-APP^{695WT} cells and primary neurons were performed following the same protocol described above. At the end of treatments, cells were fixed with 4% paraformaldehyde in PBS during 15 min at rt and rinsed 3 times 5 min with PBS. Cells were then permeabilized using 0.25% Triton X-100 during 15 min at rt and rinsed with PBS 3 times for 5 min before blocking in 1% BSA in PBS for 1 h at rt. After 3 washes with PBS, cells were incubated with primary antibodies directed against LC3-B (1/200) and p62 (1/200) at 4° C. overnight in a PBS solution containing 1% BSA and 0.02% Triton X-100. The following day, cells were rinsed with PBS and incubated with anti-IgG secondary antibodies coupled to Alexa Fluor 568 (1/500) (Invitrogen).

Nuclei were visualized using DAPI (Thermo Scientific). Images were acquired on a Zeiss Axio Imager Z2 microscope (Carl Zeiss, Germany) equipped with a Hamamatsu ORCA-Flash4.0 digital camera (Hamamatsu Photonics, Japan) and ApoTome.2 system (Carl Zeiss, Germany). For Apotome image acquisition, the Axio Imager was used with optical plan-apochromat 63×/1.4 oil M27 at rt.

β-secretase in vitro activity assay. These experiments were performed on human recombinant BACE-1 protein at Eurofins Cerep (Celle-Lévescault, France), with minor variations to the experimental protocol described by Ermolieff, J. et al. The cleavage of fluorescent Mca-S-E-V-N-L-D-AE-F-R-K(Dnp)-R-R-NH₂ substrate to Mca-S-E-V-N-L-NH₂ was monitored after 60 min incubation at rt in the presence or absence of test molecules. Compounds 30 and 31 were evaluated at concentrations of 1 and 10 μM. Both compounds were found totally inactive (% inhibition <9%). Under the assay conditions, reference OM 99-2 displayed an IC₅₀ value of 59 nM.

Statistical Analysis. For statistical analysis, the non-parametric Mann-Whitney test was selected using GraphPad Prism 6 (GraphPad Software) and statistical significance was set at *p<0.05, **p<0.001, ***p<0.001 and ****p<0,0001. All data are reported as mean±SEM.

### Results

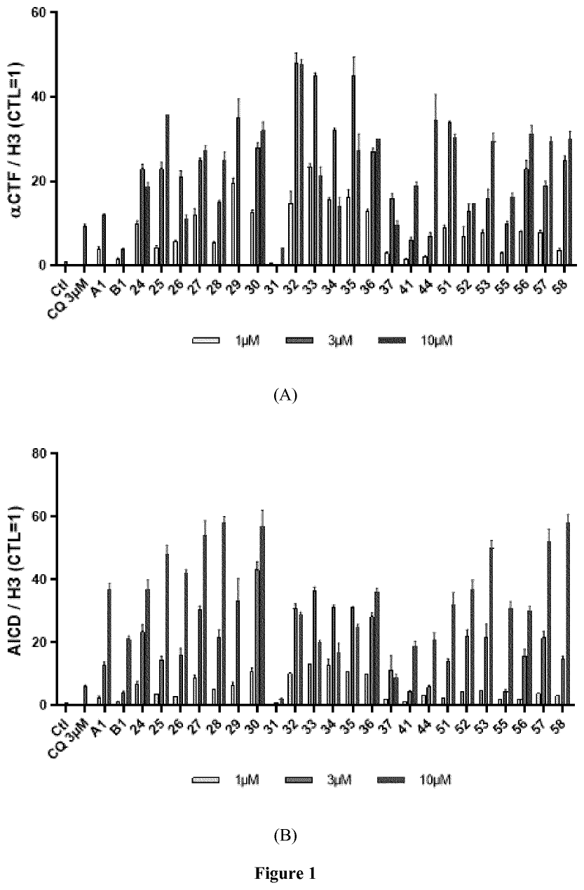

Compounds of the invention (compounds 24-37, 41, 44 and 51-58) were evaluated for their ability to modulate APP processing on SY5Y human neuroblastoma cell line stably expressing the neuronal isoform of human wild-type APP695 (SYSY-APPwt). Aβ levels (Aβ₁₋₄₀ and Aβ₁₋₄₂) after treatment with reference and tested compounds were measured by ELISA (Table 2). Results are expressed as IC₅₀ values which correspond to the concentration of a given compound that inhibits Aβ secretion by 50% (either Aβ₁₋₄₀ or Aβ₁₋₄₂). APP carboxy-terminal fragments (αCTFs and AICD) were measured by Western blot and quantified. The effect of reference and tested compounds on αCTFs and AICD are expressed as the intensity of corresponding western-blot bands resulting from the secretase cleavages. This effect is measured at various concentrations (1, 3, 10 μM, FIG. 1) and compared to the one of chloroquine (CQ) at 3 μM (Table 2).

TABLE 2
Effects of compounds of the invention on Aβ₁₋₄₀ and Aβ₁₋₄₂ secretion

| | | | CTF_{α} | AICD |
|---|---|---|---|---|
| | Aβ₁₋₄₀ | Aβ₁₋₄₂ | vs CQ | vs CQ |
| Compound | IC₅₀ (μM)^{a} | IC₅₀ (μM)^{a} | (3 μM)^{b,c} | (3 μM)^{b,d} |
| CQ | 7.0 | 12.7 | 1 | 1 |
| 24 | 2.1 ± 0.6 | 3.3 ± 2.1 | 2.6 | 2.5 |
| 25 | 1.5 ± 0.1 | 1.7 ± 0.4 | 2.1 | 2.5 |
| 26 | 4.3 ± 0.7 | 4.8 ± 0.6 | 3.3 | 2.6 |
| 27 | 2.6 ± 0.1 | 4.1 ± 1.3 | 4.3 | 2.7 |
| 28 | 2.8 ± 1.2 | 4.2 ± 1.5 | 3.1 | 1.7 |
| 29 | 2.6 ± 0.2 | 2.7 ± 0.8 | 5.1 | 3.7 |
| 30 | 2.8 ± 1.2 | 3.4 ± 1.9 | 6.4 | 3.6 |
| 31 | 2.1 ± 0.3 | 2.8 ± 0.1 | 0.1 | 0.1 |
| 32 | 2.1 ± 0.3 | 2.0 ± 0.1 | 4.5 | 5.1 |
| 33 | 1.4 ± 0.5 | 1.2 ± 0.6 | 5.0 | 4.8 |
| 34 | 2.0 ± 0.2 | 1.8 ± 0.7 | 4.5 | 3.4 |
| 35 | 1.6 ± 0.1 | 1.6 ± 0.3 | 4.4 | 4.8 |
| 36 | 1.7 ± 0.3 | 1.6 ± 0.8 | 4.2 | 3.0 |
| 37 | 5.2 ± 0.1 | 4.0 ± 0.4 | 2.4 | 2.1 |
| 41 | 1.5 ± 0.3 | 1.2 ± 0.3 | 0.6 | 0.7 |
| 44 | 3.9 ± 1.0 | 3.6 ± 0.1 | 0.9 | 0.8 |
| 51 | 5.0 ± 0.9 | 6.5 ± 2.0 | 3.6 | 2.0 |
| 52 | 3.7 ± 1.3 | 5.0 ± 0.3 | 3.3 | 1.2 |
| 53 | 3.3 ± 0.2 | 3.5 ± 1.1 | 1.7 | 3.3 |
| 54 | 8.3 ± 4.0 | 12.1 ± 7.8 | nd | |
| 55 | 6.1 ± 0.7 | 8.0 ± 1.6 | 1.2 | 0.8 |
| 56 | 5.3 ± 0.1 | 6.3 ± 0.7 | 2.5 | 2.7 |
| 57 | 2.8 ± 1.1 | 3.4 ± 2.0 | 2.2 | 2.6 |
| 58 | 5.1 ± 1.3 | 5.4 ± 0.4 | 3.0 | 1.8 |

^{a} Compound concentration inhibiting 50% of Aβ1-40 or Aβ1-42 peptide secretion in SY5Y cells. IC₅₀ values are expressed as mean ± SD of at least two experiments performed in triplicate.
^{b}Mean values calculated on the basis of at least three independent experiments with less than 10% deviation.
^{c}CTFα increase compared to chloroquine ([CTF]compound/[CTF]CQ) at 3 μM.
^{d}AICD increase compared to chloroquine ([AICD]compound/[AICD]CQ) at 3 μM.
nd: not determined

Results show that compounds of the invention were able to decrease Aβ secretion and to promote AICD and αCTFs stability.

### In Vivo Evaluations

### Materials and Methods

Thy-Tau22 transgenic colonies (C57Bl/6J genetic background) were obtained by crossing heterozygous males C57Bl/6J with WT females. All animals were housed in a pathogen-free facility at 5 to 6 animals per cage (Techniplast Cages 1284L), with ad libitum access to food and water in a 12/12-hour light-dark cycle and maintained under a constant temperature of 22° C. For treatment, animals were randomly distributed and compound 30 or 31 was provided in the drinking water at a final concentration of 1 mg/kg, ie 12.5 μg/mL for drinking solutions taking into account an average weight of 25 g/mouse drinking 4 mL/day. Drinking bottles were changed once per week as aqueous solutions of compounds 30 and 31 were previously demonstrated to be stable during more than one week, and the volume consumed was measured throughout the treatment period. Food consumption and bodyweight were also assessed. In WT animals, a pilot study of drug treatment was performed for one month to establish the innocuousness of compound 30 and 31 treatments. Thy-Tau22 females were treated for 1 months, starting at 6 months of age.

Before any behavioral test, exploratory behavior and locomotion were evaluated in treated and untreated animals in an Openfield (OF) 25 cm×25 cm arena. One acquisition in 2 joined arenas were performed simultaneously. Each mouse were placed in the arena and allowed to explore for 10 minutes. Parameters including distance, speed and velocity were acquired by video recording using EthoVision video tracking equipment and software (Noldus Information Technology, Paris, France) in a dedicated room. Anxiety, which could interfere with the memory test, was evaluated using the elevated plus maze (EPM). Mice were placed in the center of a plus-shaped maze consisting of two 10-cm-wide open arms and two enclosed arms elevated to 50 cm from the floor. Locomotion, distance, speed and velocity were measured, as well as the number of entries into each arm, time spent in open versus closed arms, percentage of open arm entries and time spent in the open arms during a 5 minutes test.

Y-Maze: Short-term memory was tested on a Y-maze. The Y-maze consists of three enclosed arms surrounded by spatial clues. One arm was closed off during the learning phase. Each mouse was positioned in the Starting arm. Mice were allowed to explore the maze for 5 min. During 2 min of retention time, the closed arm was opened and the mouse was re-placed in the starting arm. The previously closed armed was named the «new arm» (N) and the third arm was named the «other arm» (0). Parameters—total distance travelled, velocity, alternation between the arms, entries into the new, starting or other arm—were measured over 5 minutes. The short-term memory test was considered successful when the proportion of entries into the new arm was higher than into the other two.

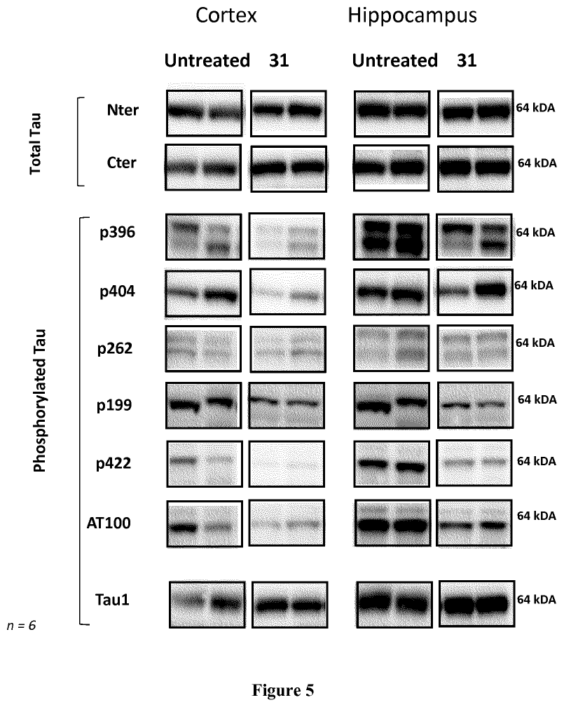

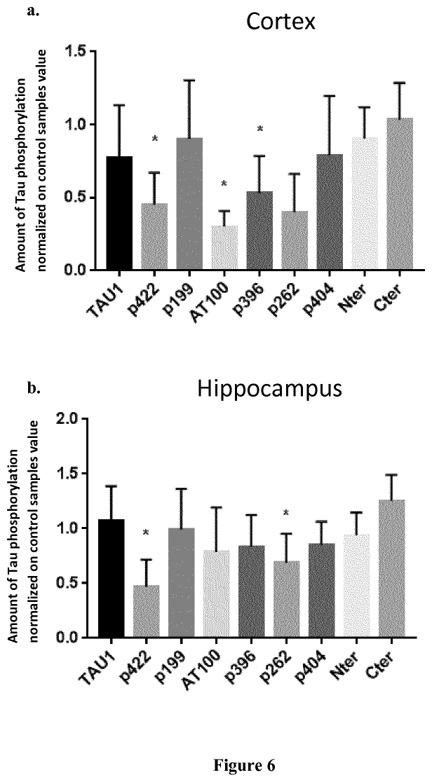

Hyperphosphorylation and phosphorylation levels of Tau. Tau expression, phosphorylation and hyperphosphorylation in the hippocampus and in the cortex were investigated by Western-blotting (FIGS. 5 and 6). The mice were euthanized by beheading and their brain was dissected and was put for few seconds in an isopropanol solution for post-mortem conservation. The samples were sonicated with a Tris-Sucrose Buffer during 30s and were prepared for Western-blot analysis by adding 1 volume of NuPage NuPAGER©LDS 2× sample buffer supplemented with 20% NuPAGER© sample reducing agents (Invitrogen). Samples were heated 10 min at 70° C. 8 μg of proteins per well were loaded onto precast 12% Criterion XT Bis-Tris polyacrylamide 26 wells gels (Bio-Rad) and electrophoresis was achieved by applying a tension of 200V during 60 min using a Criterion electrophoresis Cell with the NuPAGER©MOPS SDS running buffer (1×). Proteins were transferred to a nitrocellulose membrane of 0.404 pore size (G&E Healthcare) using the Criterion blotting system by applying a tension of 100 V for 40 min. Molecular weights calibration was achieved using molecular weight markers (Novex and Magic Marks, Life Technologies). Protein transfer and quality were determined by a reversible Ponceau Red coloration (0.2% Xylidine Ponceau Red and 3% Trichloroacetic acid). Membranes were then blocked in 25 mM Tris-HCl pH 8.0, 150 mM NaCl, 0.1% Tween-20 (v/v) (TBS-T) and 5% (w/v) of skimmed milk (TBS-M) or 5% (w/v) of bovine serum albumin (TBS-BSA) depending on the antibody during 1 hour. Membranes were rinsed three-times 10 min in TBS-T before incubation with primary antibodies overnight at 4° C. Membranes were rinsed 3 times 10 min with TBS-T and then incubated with secondary antibodies for 45 min at room temperature (RT). The immunoreactive complexes were revealed using a standard ECL detection procedure. Quantifications of protein expression levels were performed with ImageQuantTL Software. The results obtained with the quantification of the treated samples were divided by the results obtained with the quantification of the housekeeping gene (GAPDH) in order to check if the amount of proteins was homogeneous in each well. The obtained results for treated samples were then divided by the results for control samples, i.e. samples from untreated ThyTau22 mice, in order to normalize the data.

### Results

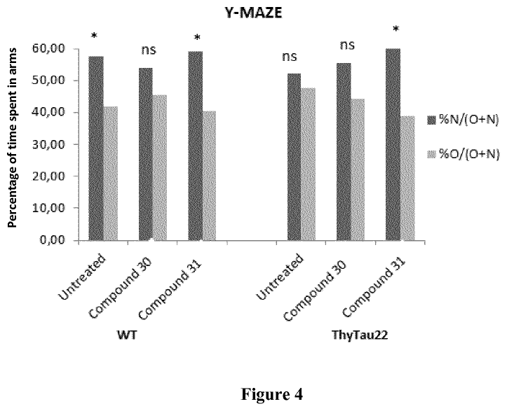

The untreated and 31-treated wild-type mice succeed in the Y-Maze behavioral task compared to the untreated ThyTau22 that clearly show a significant short-term memory deficit. Interestingly, the mice treated during one month with compound 31 show a higher short memory preservation at the Y-Maze task than the mice treated during one month with compound 30 (see FIG. 4).

The one month treatment of the ThyTau22 with compound 31 decreased the phosphorylation of Tau in the hippocampus and in the cortex. compound 31 decreased significantly the p422, AT100, p396 and p262 phosphorylation sites in the cortex and the p422 and p262 phosphorylation sites were also significantly reduced in the hippocampus of 31-treated mice (FIGS. 5 and 6), unlike the 30-treatment that showed less effect on Tau phosphorylation in the mice. Phosphorylation sites recognized with p422 and AT100 are pathological epitopes only observed when a neurofibrillary degenerating process occurs in the brain. A diminution of staining with those two antibodies is indicative of a reduction of the neurofibrillary degenerating process.

1. A compound of Formula I: 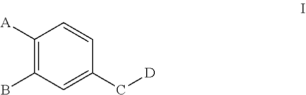
and pharmaceutically acceptable salts and solvates thereof,
wherein
A is H or a group of formula 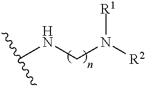
wherein
R¹ and R² are independently selected from C1-C6-alkyl and C1-C6-haloalkyl, or R¹ and R² form together with the nitrogen atom they are attached to a 6- or 7-membered heterocyclyl group, which optionally contains one or more other heteroatoms, and wherein the resulting heterocyclic moiety is optionally substituted by one or more substituents independently selected from C1-C4-alkyl, halogen and C1-C4-haloalkyl;
n is an integer from 1 to 6;
B is H or a group of formula 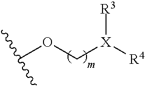
wherein
X is N or CH;
R³ and R⁴ are independently selected from C1-C6-alkyl and C1-C6-haloalkyl, or R³ and R⁴ form together with X a 6-membered cycloalkyl group or a 6- or 7-membered heterocyclyl group, which optionally contains one or more other heteroatoms, and wherein the resulting cyclic or heterocyclic moiety is optionally substituted by one or more substituents independently selected from C1-C4-alkyl, halogen and C1-C4-haloalkyl;
m is an integer from 1 to 6;
C is a 5- or 6-membered aryl or heteroaryl group;
D is H or a group of formula 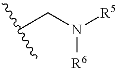
wherein
R⁵ and R⁶ are independently selected from C1-C6-alkyl, or R⁵ and R⁶ form together with the nitrogen atom they are attached to a 6- or 7-membered heterocyclyl group, which optionally contains one or more other heteroatom, and wherein the resulting heterocyclic moiety is optionally substituted by one or more substituents independently selected from C1-C4-alkyl, halogen and C1-C4-haloalkyl; and
D is located at any free position of group C;
with the proviso that at least two groups amongst groups A, B and D are not H.

2. The compound according to claim 1, wherein C is phenyl or pyrrolyl.

3. The compound according to claim 1, wherein R¹ and R² form together with the nitrogen atom they are attached to a piperidinyl group.

4. The compound according to claim 1, wherein R³ and R⁴ form together with X a cyclohexyl group or a piperidinyl group.

5. The compound according to claim 1, wherein R⁵ and R⁶ form together with the nitrogen atom they are attached to a non-aromatic 6-membered heterocyclyl group selected from piperidinyl, morpholinyl and N-methylpiperazinyl.

6. The compound according to claim 1, having Formula II: 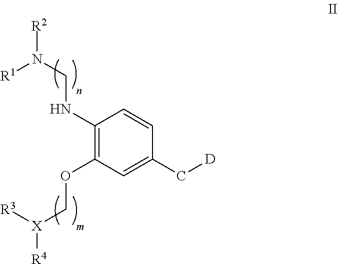
and pharmaceutically acceptable salts and solvates thereof,
wherein
X, R¹, R², R³, R⁴, n, m, C and D are as defined in claim 1,
with the proviso that D is not H when X is CH.

7. The compound according to claim 1, having Formula III: 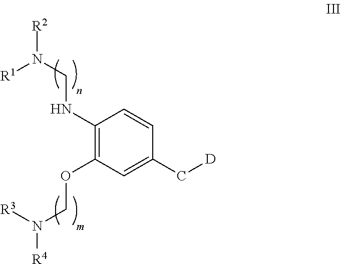
and pharmaceutically acceptable salts and solvates thereof,
wherein
R¹, R², R³, R⁴, n, m, C and D are as defined in claim 1.

8. The compound according to claim 1, having Formula IV: 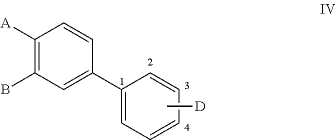
and pharmaceutically acceptable salts and solvates thereof,
wherein
A, B and D are as defined in claim 1.

9. The compound according to claim 1, having Formula V: 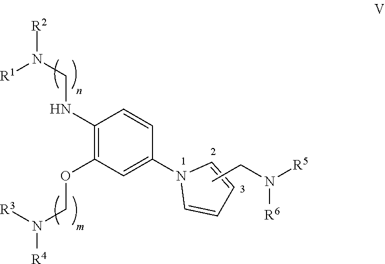
and pharmaceutically acceptable salts and solvates thereof,
wherein
R¹, R², R³, R⁴, n, m, R⁵ and R⁶ are as defined in claim 1.

10. The compound according to claim 1, selected from the group consisting of:
2-[2-(Piperidin-1-yl)ethoxy]-N-[3-(piperidin-1-yl)propyl]-4-[4-(piperidin-1-ylmethyl)phenyl]aniline,
4-(4-[(Dimethylamino)methyl]phenyl)-2-[2-(piperidin-1-yl)ethoxy]-N-[3-(piperidin-1-yl)propyl]aniline,
2-[2-(1-piperidyl)ethoxy]-4-[3-(1-piperidylmethyl)phenyl]-N-[3-(1-piperidyl)propyl]aniline,
2-[2-(Piperidin-1-yl)ethoxy]-N-[3-(piperidin-1-yl)propyl]-4-[2-(piperidin-1-ylmethyl)phenyl]aniline,
4-[2-(Morpholin-4-ylmethyl)phenyl]-2-[2-(piperidin-1-yl)ethoxy]-N-[3-(piperidin-1-yl)propyl]aniline,
4-(2-[(4-Methylpiperazin-1-yl)methyl]phenyl)-2-[2-(piperidin-1-yl)ethoxy]-N-[3-(piperidin-1-yl)propyl]aniline,
4-(2-[(Dimethylamino)methyl]phenyl)-2-[2-(piperidin-1-yl)ethoxy]-N-[3-(piperidin-1-yl) propyl]aniline,
3-(2-Cyclohexylethoxy)-2′-((dimethylamino)methyl)-N-(3-(piperidin-1-yl)propyl)-[1,1′-biphenyl]-4-amine,
2′-((Dimethylamino)methyl)-3-(3-(piperidin-1-yl)propoxy)-N-(3-(piperidin-1-yl)propyl)-[1,1′-biphenyl]-4-amine,
2′-((Dimethylamino)methyl)-3-(2-(piperidin-1-yl)ethoxy)-N-(2-(piperidin-1-yl)ethyl)-[1,1′-biphenyl]-4-amine,
2′-((Dimethylamino)methyl)-N-(2-(piperidin-1-yl)ethyl)-3-(3-(piperidin-1-yl)propoxy)-[1,1′-biphenyl]-4-amine,
2′-((Dimethylamino)methyl)-3-(4-(piperidin-1-yl)butoxy)-N-(2-(piperidin-1-yl)ethyl)-[1,1′-biphenyl]-4-amine,
2′-((Dimethylamino)methyl)-N-(4-(piperidin-1-yl)butyl)-3-(2-(piperidin-1-yl)ethoxy)-[1,1′-biphenyl]-4-amine,
3-(2-(Piperidin-1-yl)ethoxy)-N-(3-(piperidin-1-yl)propyl)-[1,1′-biphenyl]-4-amine,
N,N-Dimethyl-1-(3′-(2-(piperidin-1-yl)ethoxy)-[1,1′-biphenyl]-2-yl)methanamine,
2′-((Dimethylamino)methyl)-N-(3-(piperidin-1-yl)propyl)-[1,1′-biphenyl]-4-amine,
4-(3-[(Dimethylamino)methyl]-1H-pyrrol-1-yl)-2-[2-(piperidin-1-yl)ethoxy]-N-[3-(piperidin-1-yl)propyl]aniline,
2-[2-(Piperidin-1-yl)ethoxy]-N-[3-(piperidin-1-yl)propyl]-4-[3-(piperidin-1-ylmethyl)-1H-pyrrol-1-yl]aniline,
4-[3-(Morpholin-4-ylmethyl)-1H-pyrrol-1-yl]-2-[2-(piperidin-1-yl)ethoxy]-N-[3-(piperidin-1-yl)propyl]aniline,
4-(3-[(4-Methylpiperazin-1-yl)methyl]-1H-pyrrol-1-yl)-2-[2-(piperidin-1-yl)ethoxy]-N-[3(piperidin-1-yl)propyl]aniline,
4-(3-[(dimethylamino)methyl]-1H-pyrrol-1-yl)-N-[3-(dimethylamino)propyl]-2-[2-(piperidin-1-yl)ethoxy]aniline,
N-[3-(Dimethylamino)propyl]-2-[2-(piperidin-1-yl)ethoxy]-4-[3-(piperidin-1-ylmethyl)-1H-pyrrol-1-yl]aniline,
N-[3-(Dimethylamino)propyl]-4-[3-(morpholine-4-ylmethyl)-1H-pyrrol-1-yl]-2-[2-(piperidin-1-yl)ethoxy]aniline, and
N-[3-(dimethylamino)propyl]-4-(3-[(4-methylpiperazin-1-yl)methyl]-1H-pyrrol-1-yl}-2-[2-(piperidin-1-yl)ethoxy]aniline.

11. A pharmaceutical composition comprising a compound according to claim 1 or a pharmaceutically acceptable salt or solvate thereof and at least one pharmaceutically acceptable carrier, diluent, excipient and/or adjuvant.

12. A medicament comprising a compound according to claim 1 or a pharmaceutically acceptable salt or solvate thereof.

13. A method of treating and/or preventing a disease involving formation of amyloid plaques and/or where a dysfunction of the APP metabolism occurs comprising the administration of a therapeutically effective amount of a compound according to claim 1 or pharmaceutically acceptable salt or solvate of the invention, to a patient in need thereof.

14. The method according to claim 13, wherein the disease involving formation of amyloid plaques and/or where a dysfunction of the APP metabolism occurs is Alzheimer's disease.

15. (canceled)

16. A method for modulating Aβ peptides production and/or stabilizing αCTFs and AICD expression in a patient in need of such treatment, which comprises administering to said patient an effective amount of a compound according to claim 1 or a pharmaceutically acceptable salt or solvate thereof.

17. A method for inhibiting Aβ peptides production and/or stabilizing αCTFs and AICD expression in a patient in need of such treatment, which comprises administering to said patient an effective amount of a compound according to claim 1 or a pharmaceutically acceptable salt or solvate thereof.
